(19) United States
(12) Patent Application Publication
(10) Pub. No.: US 2017/0325892 A1
(43) Pub. Date: Nov. 16, 2017

(54) PATIENT-SPECIFIC PROSTHESIS ALIGNMENT

(71) Applicant: Arthromeda, Inc., Lowell, MA (US)

(72) Inventor: Mehran S. Aghazadeh, Newton, MA (US)

(21) Appl. No.: 15/593,031

(22) Filed: May 11, 2017

Related U.S. Application Data
(60) Provisional application No. 62/334,647, filed on May 11, 2016.

(51) Int. Cl. A61B 34/10; A61B 34/20; A61F 2/34; A61F 2/46; A61B 17/88; A61B 90/00; A61F 2/30; A61B 17/00

(52) U.S. Cl. CPC A61B 34/10; A61B 17/8897; A61B 34/20; A61F 2/34; A61F 2/4609; A61B 2034/2055; A61B 2017/00526; A61B 2034/108; A61B 2090/3945; A61F 2002/4681; A61B 2034/2072; A61F 2002/30948; A61F 2002/30985; A61B 2090/373

(57) ABSTRACT

Systems and methods for providing alignment of instruments and/or prostheses in various surgical operations are provided herein. The systems and methods generally include one or more sensors coupled to a patient's bones or other surgical tools, the sensors can detect their position and orientation in space and communicate this information to a processor. The processor can utilize the information to display data to a surgeon or other user regarding the position, angle, and alignment of a patient's bones, surgical tools, and prostheses. Further, the one or more sensors can be aligned to the patient's anatomy using a patient-specific alignment guide that interfaces with a portion of the patient's anatomy in a single position/orientation.

### RELATED APPLICATION

This application claims benefit under 35 U.S.C. §119(e) of U.S. Provisional Patent Application No. 62/334,647, filed May 11, 2016, and incorporated by reference in its entirety herein.

### FIELD

This disclosure generally relates to surgical procedures and, more particularly, to systems and methods utilized in prosthetic implantation surgeries.

### BACKGROUND

Successful prosthetic surgery requires precise intra-operative placement and positioning of replacement structures as implants within the patient such that the in vivo function of the reconstructed joint is optimized biomechanically and biologically. For the surgeon, it is necessary to ensure that the replacement structural components are implanted correctly and function in situ properly in order to avoid intra-operative and post-operative complications, as well as to ensure a long-lasting action and use for the implanted prosthesis.

While applicable to any prosthetic implant, one example where placement and positioning are important is hip arthroplasty. In such an operation, a malpositioned hip prosthesis will not adequately restore the joint's biomechanics, will not function properly, and is at increased risk of intra-operative and post-operative complications. Such complications can include, without limitation, dislocation, impingement, fracture, implant failure, aseptic loosening, and subsidence. A malpositioned prosthetic implant is particularly susceptible to dislocation and early loosening because the prosthesis will not be well fitted or supported within the host's native bone.

One problem routinely faced by surgeons in human hip replacement procedures is how to achieve proper acetabular prosthetic implant alignment. It is generally agreed among orthopedic surgeons that the ideal anatomic position (for most patients) for an acetabular prosthetic implant within the native bone of the host's hip is at 45° (degrees) of inclination. Despite this general agreement, however, surgeons often select different desired angles of inclination based on the particular anatomy of a given patient.

A second important angle is the angle of forward flexion. The range of optimum angles of forward flexion can vary widely based on a patient's anatomy, biomechanics, and flexibility of different joints, among other factors. As a result and similar to inclination as described above, surgeons often select different angles of forward flexion for different patients. More recent techniques emphasize “combined anteversion” of a reconstructed hip, rather than a prosthetic cup's absolute angle of forward flexion. Combined anteversion is the sum of the angle of forward flexion of the cup and the angle of anteversion of a stem that is fitted into a patient's femur. Since there is limited space for changing the stem's angle of anteversion, adjusting the position of the cup to that of the stem is critical to improving stability of the reconstructed hip and reducing impingement.

Precise measurement of these specific angles, and therefore proper placement of the prosthesis, has been difficult to achieve, mostly because two of these angles are relative to the patient's pelvis and the patient is covered by sterile surgical drapes during the course of a hip replacement operation. It also can be difficult to monitor any change in position of the patient's pelvis that can occur after draping the patient for the surgery, including, for example, any change in position that may occur during the operation.

Prior techniques for addressing these issues have included the use of electronic position sensors, e.g., one coupled to a patient's pelvis and the other to an instrument used to position a prosthesis, to monitor positioning of the patient relative to the instrument and prosthesis coupled thereto. In order to accurately determine the critical angles mentioned above, however, the electronic position sensor coupled to the pelvis has to be aligned with anatomical planes of the patient's body in a known manner. To accomplish this, a guide is required to orient the electronic position sensor prior to attachment to the patient's pelvis. Taking time to properly use the guide increases the overall time and cost associated with the operation and introduces the possibility of surgeon error in orienting the guide properly relative to the patient.

A recent development in the orthopedics field is the use of patient-specific components during surgical procedures. Such components can include a prosthesis itself or a guide for placing another component, making a cut in tissue or bone, etc. Patient-specific components can be created prior to an operation from a 3-dimensional model of a patient's anatomy and can be configured to interface with a portion of the patient's anatomy in only one orientation. Such a component can therefore be less prone to user error, as it fits against or into a patient's body in only one correct manner. Further, the use of such components can reduce the time required to perform an operation, as they can be quickly and accurately positioned during an operation and all work necessary to create the component can be performed well in advance of an operation.

Accordingly, there is a need for improved prosthesis positioning and alignment systems and methods that streamline surgical procedures while maximizing accuracy and ease of use. More particularly, there is a need for such systems that take advantage of patient-specific components to streamline arthroplasty procedures, such as hip, knee, and other joint replacement procedures.

### SUMMARY

The present disclosure provides systems and methods for prosthesis positioning and alignment that address the shortcomings of prior techniques described above. These systems and methods can find application in a variety of surgical procedures, such as hip arthroplasty, etc. The systems and methods described herein can provide valuable advantages relative to prior techniques, including increased accuracy, increased ease of use, reduced procedure time, reduced procedure complexity, and reduced procedure cost. This disclosure can provide systems and methods for patient-specific prosthesis alignment and positioning. The systems and methods described herein can make use of one or more electronic position sensors and a patient-specific alignment guide to accurately and quickly position a prosthesis during, for example, an arthroplasty procedure. The systems and methods described herein can utilize a patient-specific alignment guide to provide registration relative to anatomical planes of the patient's body. Because the patient-specific alignment guide can be created in software and fabricated prior to a surgical procedure, time required in an operating room can be reduced. Moreover, any possible error that might occur due to a surgeon or other user incorrectly positioning an electronic sensor using, e.g., an axis guide, can be eliminated.

In one aspect, a system for use in implanting a prosthesis is provided that can include a digital data processor, a display, first and second electronic position sensors, a patient-specific alignment guide, and application software. The first and second electronic position sensors can be capable of reporting information about their respective position and orientation in 3-dimensional space to the digital data processor. The patient-specific alignment guide can be configured to interface with a bony anatomic structure of a patient. The alignment guide can include a surface created from a scan of the patient's bony anatomic structure that is substantially a negative of at least a portion of the patient's bony anatomic structure. The application software can (i) receive information from the first electronic position sensor, (ii) receive information from the second electronic position sensor, and (iii) calculate and display angular relationships derived from the information received from the first and second electronic position sensors.

The systems and methods described herein can include any of a variety of additional or alternative features and/or components, all of which are considered within the scope of the present disclosure. For example, the first electronic position sensor can be configured to be coupled to the patient's bony anatomic structure and the second electronic position sensor can be configured to be coupled to the patient-specific alignment guide. In some embodiments, at least one of the first electronic position sensor and the second electronic position sensor can be configured to communicate wirelessly with the digital data processor. In other embodiments, the first electronic position sensor and the second electronic position sensor can be wired together, and the second electronic position sensor can be configured to communicate with the first electronic position sensor through the wire.

In certain embodiments, the bony anatomic structure can be a pelvis. In such embodiments, the angular relationships can include any of (pelvic) axial tilt, (pelvic) anterior-posterior (AP) tilt, absolute angle of inclination, absolute angle of forward flexion, true angle of inclination, and true angle of forward flexion. In some embodiments, the calculation can be performed on demand. In other embodiments, the calculation can be performed continuously in real time.

In certain other embodiments, the first electronic position sensor can include a leg length measurement sensor configured to detect a distance between the first electronic position sensor and the second electronic position sensor. In some embodiments, such a sensor can include a laser and a receiver. In such an embodiment, the software can be configured to determine the distance from the first electronic position sensor to the second electronic position sensor by activating the laser and detecting the laser with the receiver after it is reflected off another surface, e.g., a surface of the second electronic position sensor.

In still further embodiments, the first electronic position sensor can include a laser emitter and the second electronic position sensor can include a receiver. In such an embodiment, the software can be configured to determine an offset between the first and second electronic position sensors based on which portion of the receiver detects light from the laser emitter.

In another aspect, a method for creating a patient-specific alignment guide is provided. The method can include receiving data from a scan of a bony anatomic structure of a patient and creating a 3-dimensional model of the patient's bony anatomic structure from the data. The method can further include creating a 3-dimensional model of a patient-specific alignment guide including a surface that is substantially a negative of at least a portion of the patient's bony anatomic structure and fabricating the patient-specific alignment guide.

As with the system described above, the method can include any of a variety of additional or alternative steps or features. For example, the bony anatomic structure can be a pelvis. Further, the method can include determining at least one of an angle of inclination and an angle of forward flexion of the patient-specific alignment guide relative to the patient's pelvis. In some embodiments fabricating the patient-specific alignment guide can utilize additive manufacturing. In other embodiments, the data received can be a series of 2-dimensional computed tomography (CT) images.

In yet another aspect, a method for positioning a prosthesis is provided. The method can include coupling a first electronic position sensor to a bony anatomic structure of a patient. The method can further include positioning a patient-specific alignment guide against the bony anatomic structure of the patient. The patient-specific alignment guide can include a surface created from a scan of the patient's bony anatomic structure that is substantially a negative of at least a portion of the patient's bony anatomic structure such that the alignment guide fits against the bony anatomic structure of the patient in only one orientation. The method can also include coupling a second electronic position sensor to the patient-specific alignment guide and communicating position and orientation information from each of the first and second electronic position sensors to a digital data processor. The method can additionally include removing the patient-specific alignment guide and coupling the second electronic position sensor to an instrument that is coupled to a prosthesis. The method can further include displaying one or more calculated angular relationships of the prosthesis relative to the patient's bony anatomic structure based on position and orientation information communicated from each of the first and second electronic position sensors and positioning the prosthesis relative to the bony anatomic structure of the patient such that the one or more displayed angular relationships are at desired levels.

In some embodiments, coupling the first electronic position sensor to the patient's pelvis can include driving two pins into the bony anatomic structure in a parallel orientation and sliding two through-holes formed in the first electronic position sensor over the two pins. In other embodiments, coupling the first electronic position sensor to the patient's pelvis can include driving one pin into the bony anatomic structure and sliding one through-hole formed in the first electronic position sensor over the pin. In still other embodiments, the method can additionally include saving position and orientation information communicated from each of the first and second electronic position sensors when the patient-specific alignment guide is positioned against the bony anatomic structure, and providing guidance to a user to direct the second electronic position sensor to the saved position relative to the first electronic position sensor when the second electronic position sensor is coupled to the instrument and after the patient-specific alignment guide has been removed.

In certain embodiments, the method can further include removably securing the patient-specific alignment guide against the bony anatomic structure using at least one surgical pin. In other embodiments, the method can include coupling a sensor mount to the instrument. In such embodiments, the sensor mount can be configured to orient the second electronic position sensor at an angle of about 45° relative to a longitudinal axis of the instrument.

In certain other embodiments, the bony anatomic structure can be a pelvis. In such embodiments, the prosthesis can be configured to be inserted into an acetabulum of the patient's pelvis.

In still another aspect, a method for positioning a prosthesis is provided. The method can include coupling a first electronic position sensor to a bony anatomic structure of a patient and coupling a second electronic position sensor to an instrument that is coupled to a prosthesis. The method can further include positioning the prosthesis relative to the bony anatomic structure of the patient and communicating position and orientation information from each of the first and second electronic position sensors to a digital data processor. The method can also include displaying one or more calculated angular relationships of the prosthesis relative to the patient's bony anatomic structure based on position and orientation information communicated from each of the first and second electronic position sensors, receiving by the digital data processor patient-specific information including one or more desired angular relationships, and adjusting the position of the prosthesis relative to the bony anatomic structure of the patient such that the one or more displayed angular relationships match the desired angular relationships.

In certain embodiments, the patient-specific information received by the digital data processor can be derived from one or more x-ray images taken intra-operatively after positioning the prosthesis relative to the bony anatomic structure of the patient.

While the above summary lists certain features, combinations, and variations of the systems and methods described herein, it is not exhaustive. Any of the features or variations described above can be applied to any particular aspect or embodiment of the disclosure in a number of different combinations. The absence of explicit recitation of any particular combination is due solely to the avoidance of repetition in this summary.

### BRIEF DESCRIPTION OF THE FIGURES

FIG. 1 illustrates various anatomical planes and axes of the human body;

FIG. 2 illustrates one embodiment of a system for use in performing hip arthroplasty;

FIG. 3 illustrates one embodiment of a method for creating a patient-specific alignment guide;

FIG. 4 illustrates one embodiment of a patient-specific alignment guide;

FIG. 5 illustrates another embodiment of a patient-specific alignment guide;

FIG. 6 illustrates still another embodiment of a patient-specific alignment guide;

FIG. 7 illustrates one embodiment of a method for positioning a hip prosthesis;

FIG. 8 illustrates one embodiment of a patient's pelvis with surgical pins for coupling an electronic position sensor thereto;

FIG. 9 illustrates one embodiment of a pin alignment guide;

FIG. 10 illustrates one embodiment of first and second electronic position sensors being synced;

FIG. 11 illustrates one embodiment of an electronic position sensor;

FIG. 12 illustrates one embodiment of a patient-specific alignment guide positioned against an acetabulum;

FIG. 13 illustrates one embodiment of a patient-specific alignment guide, first electronic position sensor, and second electronic position sensor establishing a reference position;

FIG. 14 illustrates one embodiment of first and second electronic position sensors being positioned following removal of a patient-specific alignment guide;

FIG. 15 illustrates one embodiment of a hip prosthesis being positioned by an instrument with a second electronic position sensor coupled thereto;

FIG. 16A illustrates one embodiment of a sensor mount that can be used to couple a second electronic position sensor to an instrument used to position a hip prosthesis;

FIG. 16B illustrates an alternative embodiment of a sensor mount that can be used to couple a second electronic position sensor to an instrument used to position a hip prosthesis;

FIG. 17 illustrates one embodiment of a display that can guide positioning of a hip prosthesis using position information from first and second electronic position sensors;

FIG. 18 illustrates the display of FIG. 17 in greater detail;

FIG. 19 illustrates one embodiment of a method for positioning a prosthesis;

FIG. 20A illustrates one embodiment of a system for use in performing hip arthroplasty;

FIG. 20B illustrates an alternative embodiment of a system for use in performing hip arthroplasty; and

FIG. 21 illustrates still another embodiment of a system for use in performing hip arthroplasty.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. Those skilled in the art will understand that the devices, systems, and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. To the extent that features are described herein as being a “first feature” or a “second feature,” such numerical ordering is generally arbitrary, and thus such numbering can be interchangeable. Further, in the present disclosure, like-numbered components of the embodiments generally have similar features, and thus within a particular embodiment each feature of each like-numbered component is not necessarily fully elaborated upon. Sizes and shapes of the systems and devices, and the components thereof, can depend on a number of factors, including, for example, the anatomy of a subject with which the systems and devices will be used, the size and shape of components with which the systems and devices will be used, and the methods and procedures in which the systems and devices will be used. Additionally, to the extent that linear or circular dimensions are used in the description of the disclosed devices, systems, and methods, such dimensions are not intended to limit the types of shapes that can be used in conjunction with such instruments and methods. A person skilled in the art will recognize that an equivalent to such linear and circular dimensions can easily be determined for any geometric shape. The figures provided herein are not necessarily to scale.

This disclosure provides systems and methods for patient-specific prosthesis alignment and positioning. The systems and methods described herein make use of a plurality of electronic position sensors and a patient-specific alignment guide to accurately and quickly position a prosthesis during, for example, an arthroplasty procedure. The electronic position sensors are small, inexpensive, and accurate devices similar to those described in U.S. patent application Ser. No. 14/346,632, filed Mar. 21, 2014, entitled “SYSTEM AND METHOD FOR PRECISE PROSTHESIS POSITIONING IN HIP ARTHROPLASTY” (Atty. Docket No. 112428-19); Ser. No. 14/330,261, filed Jul. 14, 2014, entitled “SENSOR FOR MEASURING THE TILT OF A PATIENT'S PELVIC AXIS” (Atty. Docket No. 112428-21); and Ser. No. 14/331,149, filed Jul. 14, 2014, entitled “SYSTEMS AND METHODS FOR ALIGNING A MEDICAL DEVICE WITH A PELVIC AXIS” (Atty. Docket No. 112428-22). The entire contents of each of these applications are incorporated by reference herein.

In contrast to certain embodiments of the systems and methods in the above-referenced applications, the systems and methods provided herein do not require the use of an axis guide to precisely position an electronic position sensor relative to one or more anatomical planes of a patient's body. Rather, the systems and methods described herein utilize a patient-specific alignment guide to provide registration relative to anatomical planes of the patient's body. Because the patient-specific alignment guide can be created in software and fabricated prior to a surgical procedure, time required in an operating room can be reduced. Moreover, any possible error that might occur due to a surgeon or other user incorrectly positioning an electronic sensor using, e.g., an axis guide, can be eliminated.

Accordingly, the systems and methods described herein can provide valuable advantages relative to prior techniques, including increased accuracy, increased ease of use, reduced procedure time, reduced procedure complexity, and reduced procedure cost.

Although many of the words, terms, and titles used herein are commonly employed and conventionally understood in their traditional medical usage and surgical context, a summary of descriptive information and definitions is presented below for some human anatomical sites, for specific medical phrases and surgical applications, and for particular jargon, designations, epithets, or appellations. These points of information, descriptions, and definitions are provided herein to avoid the misinformation, misunderstandings, and ambiguities which can exist; as an aid and guide to recognizing the particulars of the present disclosure; and for appreciating the scope and breadth of the present disclosure.

### Anatomical Planes of the Human Body:

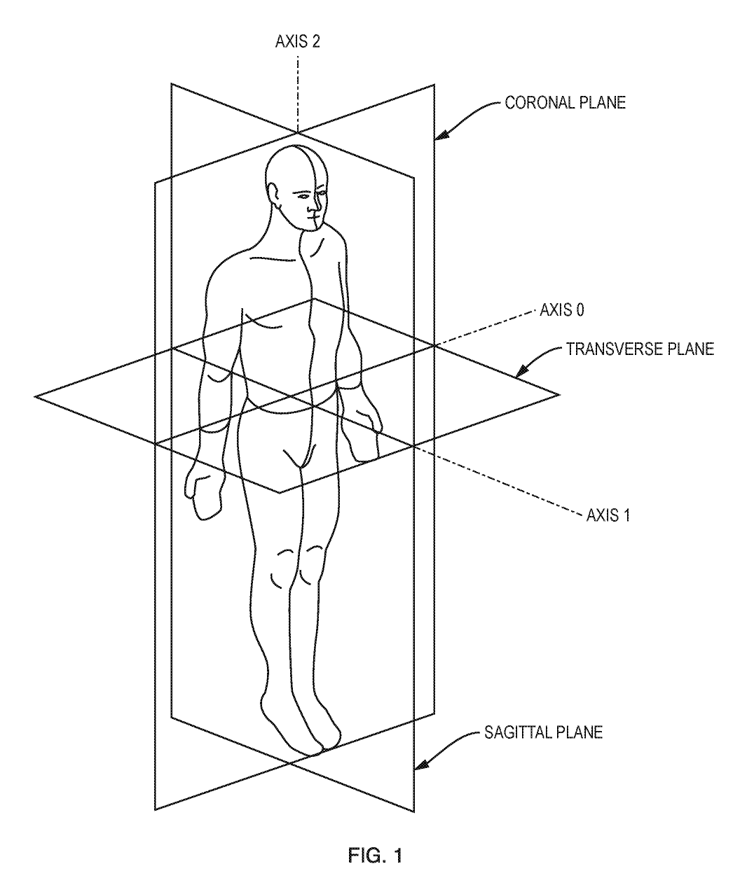

The transverse or axial plane divides the human body into top and bottom sections; the coronal plane divides the body into front (anterior) and back (posterior) portions; and the sagittal plane divides the body into left-sided and right-sided portions. Each of these anatomical planes is illustrated by FIG. 1.

Also by definition and anatomical convention, “Axis 0” is the common line between the transverse and coronal planes; “Axis 1” is the common line between the transverse and sagittal planes; and “Axis 2” is the common line between the coronal and sagittal planes.

The pelvic axis is any line defined by the pelvis and generally parallel to Axis 0 or generally perpendicular to the sagittal plane. Each of these anatomical axes is also shown in FIG. 1.

### Patient Orientation Information:

A human patient having hip joint replacement surgery is traditionally placed in the lateral decubitus position, i.e., lying down on the side opposite the surgical side. In this position, the patient's operative hip is up. Alternatively, the patient having hip joint replacement surgery can be placed in the supine position, i.e., lying on the back. Other patient positions are possible, for example, when undergoing procedures to replace other joints (e.g., knee, etc.).

The following definitions apply to the lateral decubitus position. In an ideal situation, the “pelvic axis” or “Axis 0” is perpendicular to the horizontal plane and lies parallel to the axis of gravity. Also in an ideal situation, each of Axis 1 and Axis 2 lies parallel to the horizontal plane.

In such a position, the “axial tilt” is the deviation of Axis 2 (the body's long axis) from the true horizontal plane. Axial tilt is deemed to be zero (0) when Axis 2 is parallel to the true horizontal plane. The axial tilt is assigned a positive value when the patient's head is tilted in a direction below, or his legs are tilted in a direction above, the true horizontal plane. Conversely, the axial tilt is assigned a negative value in the opposite situation, i.e., when the patient's head is tilted in a direction above, or his legs are tilted in a direction below, the true horizontal plane.

The “anterior-posterior” (or “AP”) tilt is the deviation of Axis 1 from the true horizontal plane. AP tilt is zero (0) when Axis 1 is parallel to the true horizontal plane. A forward AP tilt (rotation toward prone position) is assigned a positive value, and a backward AP tilt (rotation toward supine position) is assigned a negative value.

The following definitions apply to the supine position. In an ideal situation, the pelvic axis or Axis 0 is parallel to the horizontal plane and lies perpendicular to the axis of gravity. Also in an ideal situation, Axis 2 is parallel to the horizontal plane, as in the lateral decubitus position.

The axial tilt is the deviation of Axis 2 from the true horizontal plane. Axial tilt is deemed to be zero (0) when Axis 2 is parallel to the true horizontal plane. The axial tilt is assigned a positive value when the patient's head is tilted in a direction below, or his legs are tilted in a direction above, the true horizontal plane. Conversely, the axial tilt is assigned a negative value in the opposite situation, i.e., when the patient's head is tilted in a direction above, or his legs are tilted in a direction below, the true horizontal plane.

The “lateral tilt” is the deviation of Axis 0 from the true horizontal plane. Lateral tilt is zero (0) when Axis 0 is parallel to the true horizontal plane. Tilt toward the side of surgery is assigned a positive value, and tilt toward the opposite (non-surgical) side is assigned a negative value.

### Other Term Definitions:

The following term definitions are also employed routinely herein. When discussing hip replacement surgery, angle of inclination is the angle between the axis of the acetabulum or acetabular implant and the sagittal plane, as projected onto the coronal plane. Angle of forward flexion is the angle between the axis of the acetabulum or acetabular implant and the coronal plane, as projected onto the sagittal plane. Often this measurement is also referred to as anteversion. The angle of femoral anteversion is the angle between the axis of the femoral neck and the epicondylar axis (of the distal femur). Combined anteversion is the sum of the angles of forward flexion of the acetabular implant and the angle of femoral anteversion of the femoral implant. Epicondylar axis is a line connecting medial and lateral epicondyles of the distal femur. Angles are “absolute” angles when measured relative to the actual horizontal level. Angles are “true” angles if and when measured relative to the actual position of the patient's pelvic axis and sagittal or coronal planes, respectively. For example, “true” angles can be calculated simply by measuring and/or calculating differences between position and/or orientation data received from a plurality of sensors with no reference to an actual horizontal level.

### System Components:

Systems provided herein can include a plurality of digital position and angle sensors, a patient-specific alignment guide, and specially designed software that can execute on a computing device, such as a personal computer or tablet/portable device. With reference to a hip replacement procedure, such sensors and software together can electronically measure or calculate: (1) the bony pelvis' position while lying on the operating table during surgery by coupling one of the electronic position sensors to the pelvis such that it can track movements thereof (the need to position this sensor in any particular manner relative to anatomical planes of the patient's body can be eliminated by use of the patient-specific alignment guide because, as is explained in more detail below, the orientation of this guide relative to the anatomical planes is known when it is positioned against the portion of the patient's anatomy to which it is designed to mirror); and (2) the angles of inclination and forward flexion of the native acetabulum before and while being prepared, as well as the acetabular prosthesis while being implanted into the native bone. These measurements can be made electronically and, if desired, continuously. They can be calculated in real time and in true relationship to the living host's pelvis and body axis during preparation of the host's bone and while the prosthesis is being surgically implanted into the host's native bone structure.

The unique methodology and system described herein can provide an intra-operative surgical positioning assessment and angle determination made by referencing an orientation (that has a known relation to a patient's anatomical planes) provided by a patient-specific alignment guide. The method and system provide precise information about the angles of inclination and forward flexion of the native bony acetabulum and prosthesis for proper implantation. These measurements and calculations can be made in true relationship to the host's pelvis and body axis during the time when the surgeon is preparing the host bone and handling the prosthesis and is inserting it into the host's native bone structure.

This measuring system is fast and easy to use; it is accurate and precise in its determinations; and it is cost-efficient to operate. The system requires neither sophisticated equipment nor elaborate machinery; and is also able to directly display the prosthesis' placement during implantation, thereby eliminating any subsequent need for repositioning the cup and spending time on other less accurate alignment techniques. In short, the systems and methods described herein can considerably reduce the time required to complete the entire prosthetic implantation surgical procedure.

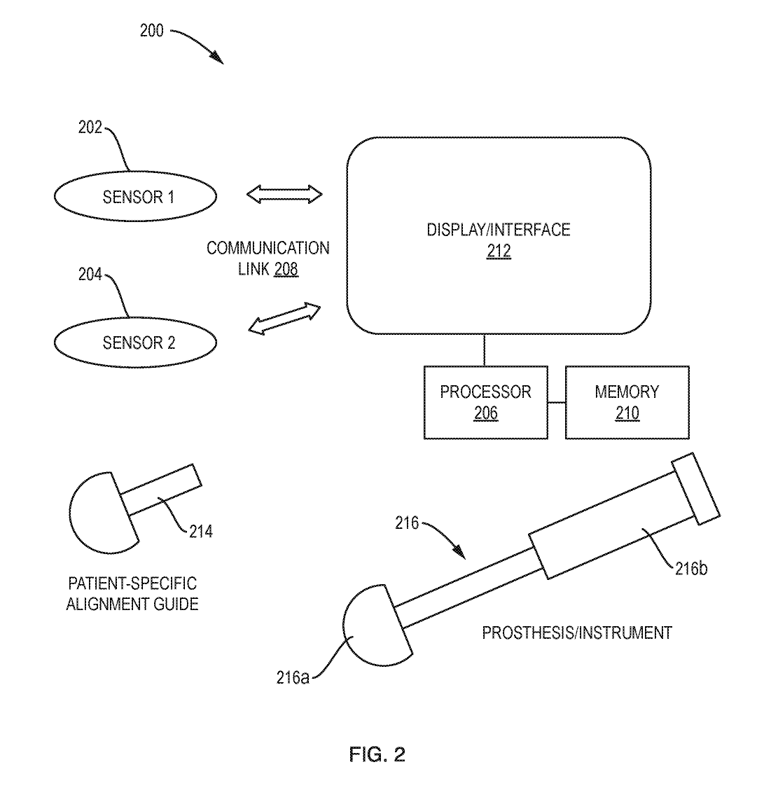

FIG. 2 illustrates one embodiment of a system according to the present disclosure. The system 200 can include a first electronic position sensor 202 and a second electronic position sensor 204. The first and second electronic position sensors 202, 204 can be configured to communicate position and/or orientation data measured thereby to a digital data processor 206 via a communication link 208. The digital data processor 206 can be part of any of a variety of computing devices, including, for example, a personal computer, tablet or other portable computing device, etc. The communication link 208 can be any of a variety of communication methods known in the art, including, for example, wired or wireless communication standards. The processor 206 can be coupled to a memory or digital data storage unit 210 that can be used to store, among other things, intra-operative software, data received from the electronic position sensors, etc. The processor 206 can also be coupled to a display 212 or other user interface (e.g., an auditory interface, tactile interface, etc.) to display measurements, calculations, or other data to a user and otherwise provide feedback to a user.

While the system illustrated in FIG. 2 can be used in connection with a variety of prosthesis implantation procedures, including, for example, knee arthroplasty, shoulder arthroplasty, etc., a more detailed description is provided below with reference to a hip replacement procedure. In such a procedure, the first electronic position sensor 202 can be configured to couple to a patient's bony pelvis in order to track movements thereof. The second electronic position sensor 204 can be configured to couple to a patient-specific alignment guide 214 that, in some embodiments, can be created prior to commencing a surgical procedure. The patient-specific alignment guide 214 can be created from a scan of a patient's anatomy, e.g., the pelvis. The alignment guide 214 can have at least one surface that is substantially a negative of a portion of the patient's anatomy, such that the alignment guide 214 can only be positioned against that portion of the patient's anatomy in one orientation.

As is explained in more detail below, creating the alignment guide 214 from a scan of the patient's anatomy can provide a known reference orientation when the alignment guide is properly positioned against the patient's anatomy. By capturing position information from the first electronic position sensor when it is coupled to the pelvis and from the second electronic position sensor when it is coupled to the patient-specific alignment guide and when the alignment guide is in position against, e.g., the patient's acetabulum, calculations can be made by the processor 206 to guide the placement of a hip prosthesis, e.g., an acetabular cup implant, to a desired angle of inclination and forward flexion relative to the patient's pelvis.

In order to provide such guidance, the second electronic position sensor 204 can be further configured to separate from the patient-specific alignment guide 214 once reference data is captured with the alignment guide in place. The second electronic position sensor 204 can then be coupled to an instrument 216 used to implant a hip prosthesis, such as an acetabular cup implant. The instrument 216 illustrated in FIG. 2 can include both the acetabular cup implant 216a and the impactor 216b that is used to implant it within a patient's acetabulum.

In one alternative embodiment, a single electronic position sensor can be utilized to perform calculations to guide the placement of a hip prosthesis, e.g., an acetabular cup implant, to a desired angle of inclination and forward flexion relative to the patient's pelvis. Information can be received from the single electronic position sensor to track movement relative to a reference position established from docking the single electronic position sensor to a patient-specific alignment guide. Once the single electronic position sensor has established a frame of reference relative to the patient's pelvis, the single electronic position sensor can be moved to one of the prosthesis or instrument to determine the relative alignment of the prosthesis or instrument. This method of operation assumes that a patient's pelvis or other bony anatomy does not move during the time that the single electronic position sensor is moved from the patient-specific alignment guide to one of the prosthesis or instrument, as any movement thereof would not be tracked due to the absence of a second electronic position sensor coupled thereto. Nonetheless, this method of operation can still provide increased accuracy and precision in placing a prosthesis relative to some prior techniques, e.g., those that rely purely on surgeon estimation, etc. Further, by repeatedly transferring the single sensor between a configuration in which it is coupled to the patient's pelvis and a configuration in which it is coupled to an instrument or implant, movement of the pelvis over time can be tracked. For example, movement of the pelvis can be detected by a position change between a first time and a second time that the single sensor was coupled to the pelvis in a same location (e.g., by coupling to one or more pins, as explained herein).

The calculations referenced above can be provided by intra-operative software executed on the processor 206. The intra-operative software can be an especially designed and coded program which is operative to read the information sent from the electronic position sensors 202, 204; display the axial or AP tilt angles and/or the absolute inclination and forward flexion angle values; calculate the true angles of inclination and forward flexion; and monitor changes in any of these values. These functions and processes can be implemented in software, hardware, firmware, or any combination thereof. The processes can be implemented in one or more computer programs executing on a programmable computer including at least one digital data processor, a storage medium or memory readable by the processor (including, e.g., volatile and non-volatile memory and/or storage elements), user input devices (e.g., the sensors described above, a keyboard, a computer mouse, a joystick, a touchpad, a touchscreen, or a stylus), and one or more output devices (e.g., a computer display). Each computer program can be a set of instructions (program code) in a code module resident in the random access memory of the computer. Until required by the computer, the set of instructions can be stored in another computer memory (e.g., in a hard disk drive, or in a removable memory such as an optical disk, external hard drive, memory card, or flash drive) or stored on another computer system and downloaded via the Internet or other network.

The foregoing components, and in particular the intra-operative software, can be used in conjunction with a digital data processor, PC, or hand-held electronic device capable of running the application software. Computers of varying kinds, capacities, and features are commercially available today and commonly employed. The particular choice of computer is thus solely a personal and individual choice, as long as it is capable of running the intra-operative software. The computer processor, PC, or hand-held device can include or be connected to an electronic visual display 212, which can display the communicated and calculated measurements. For example, the visual display can be the computer's monitor or a display portion of the hand-held device.

Any or all electronic communication connections 208 can typically be provided either as a wireless mode of communication or a hard-wire manner of communication employing a Universal Serial Bus (USB) and a standard USB cable, or by any combination of wireless and hard-wired connections. For example, in one embodiment, the electronic position sensors can be wirelessly connected to the computer processor, PC, or hand-held electronic device running the application software, while the electronic visual display can be hard-wire connected to a digital data processor, PC, or hand-held electronic device running the application software. In alternative embodiments, the electronic position sensors can be wired together and, in some embodiments, can be wired to a digital data processor to eliminate a need for each electronic position sensor to include wireless communication modules and batteries.

In order to better illustrate the methods and systems of the present disclosure, a description of an exemplary procedure for hip arthroplasty is explained below. While the procedure explained below makes use of the teachings of the present disclosure, it is by no means a limiting example. It will be appreciated that there are many variations on the procedure described below that are within the scope of the disclosure. Further, and as noted above, the teachings of the present disclosure can be applied to any of a variety of procedures where a prosthesis is implanted in a patient's body. These can include, for example, arthroplasty operations in variety of anatomical locations (e.g., knee, hip, shoulder, etc.).

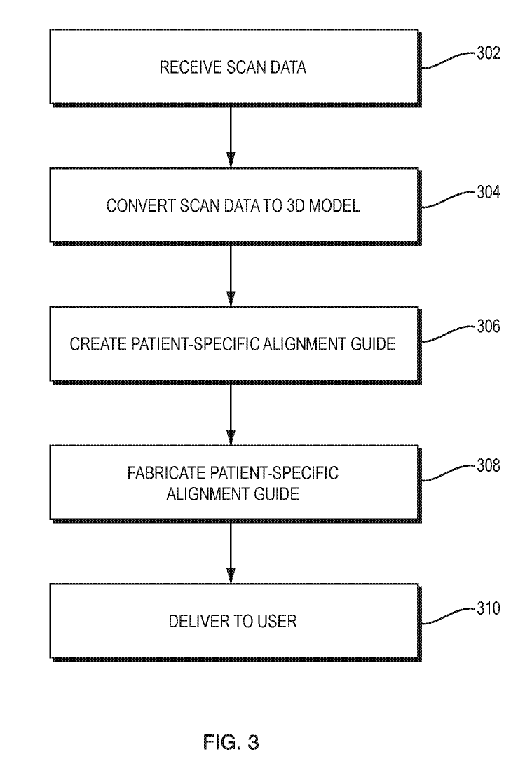

FIG. 3 illustrates one example of a pre-operative method for creating a patient-specific alignment guide, like the guide 214 shown in FIG. 2. The process generally begins by conducting a scan of at least a portion of a patient's anatomy. In the case of hip arthroplasty, such a scan can include a computed tomography (CT) scan of the patient's pelvis and acetabular region. Such a scan can produce, for example, a series of 2-dimensional images taken at different locations along an axis orthogonal to the plane defined by the images. In other embodiments, a magnetic resonance imaging (MRI) scan can be utilized.

Data from such a scan can be transferred to one or more digital data processors that are capable of running planning software to create a 3-dimensional model of the patient's pelvis from the scan data. Such a transferring process might include a variety of different technologies for relaying data, including, for example, manual transfer of physical media (e.g., a compact disc (CD), flash memory drive, or other portable storage media) or uploading scan data to one or more networked computers or storage devices. Regardless of how the transfer is accomplished, scan data can be made accessible to one or more digital data processors capable of performing the required modeling, as shown by step 302 of FIG. 3. Note that the planning software can in some embodiments be executed on the same digital data processor or computing device that is used to execute the intra-operative software (e.g., both the planning software and the intra-operative software can be part of application software running on the digital data processor or computing device), while in other embodiments separate computing devices can be utilized. In embodiments where separate devices are utilized, distributed use of the systems and methods described herein can be accomplished. For example, a patient and the users and/or digital data processors that execute the planning software can be located in different rooms, buildings, cities, states, or countries. Moreover, there can be a temporal disconnect between, for example, performing a scan of a patient and constructing a 3-dimensional model from the scan.

At step 304, the digital data processor can perform the conversion to create a 3-dimensional model of at least a portion of the patient's anatomy, e.g., a model of the patient's pelvis. As a part of this process, the model can define the anatomical planes of the patient's body based, for example, on the locations of certain anatomical landmarks. For example, for a model of a patient's pelvis, the pelvic axis (which is an axis parallel to Axis 0 of FIG. 1) can be defined by drawing a line through the left and right Anterior Superior Iliac Spine (ASIS) and other axes can be determined as orthogonal thereto. The planning software can also create any number of other axes and/or anatomical planes desired by a user. For example, in some embodiments a surgeon or other user might prefer to reference the anterior pelvic plane that is defined by the left and right ASIS and the left and right pubis. This plane, and any other plane desired by a user, can be identified and created during the planning process.

At step 306, the digital data processor (or another processor) can take the solid model of the patient's pelvis or other anatomy and create a patient-specific alignment guide in software. The patient-specific alignment guide can include at least one surface that is substantially a negative of at least a portion of the patient's anatomy such that the alignment guide can be positioned against that portion of the patient's anatomy in only one orientation. Moreover, because the anatomical—or other user defined—axes and planes of the patient's body have been determined in the 3-dimensional model, a position of the alignment guide relative to any or all of these axes and planes can be determined when the alignment guide is interfaced with its corresponding portion of the patient's anatomy. That is, the patient-specific alignment guide can provide a known reference position/orientation when interfaced with the corresponding portion of the patient's anatomy.

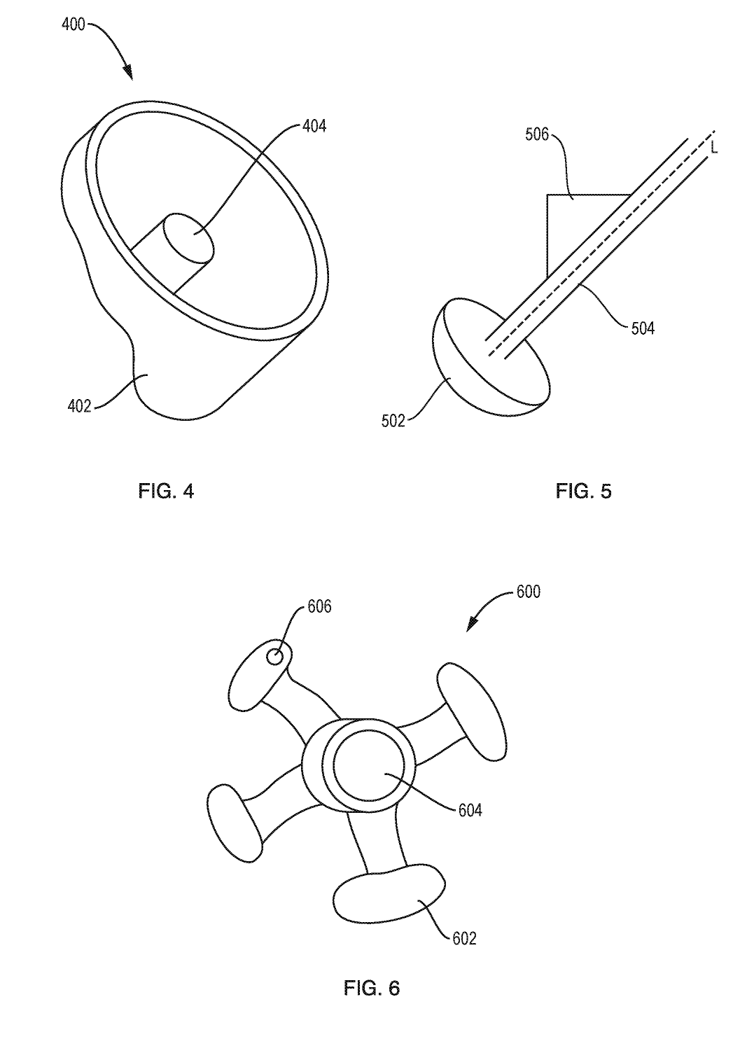

After determining the orientation of the alignment guide relative to the patient's pelvis in the 3-dimensional model, the orientation of a mounting projection (see, e.g., 404, 504, and 604 in FIGS. 4, 5, and 6, respectively) can be set according to user desire. In some embodiments, for example, the orientation of the mounting projection is not adjusted, but the values of inclination and forward flexion for the mounting projection are noted for transmission to a surgeon or other user. In some embodiments, for example, such values can be printed on the alignment guide when it is fabricated, either directly or using an encoding scheme, such as a bar code, number code, etc. These numbers can be read into the intra-operative software running on the processor 206 at the time of the surgery so that the software can accurately direct a surgeon to a desired inclination and/or forward flexion angle for an implant. In certain embodiments, information regarding the alignment guide can be transmitted to the processor 206 via a network connection, e.g., a download, rather than referencing information printed on the guide itself. For example, a surgeon or other user can enter a guide and/or patient identification code and associated data regarding the patient-specific alignment guide can be loaded into the intra-operative software.

In other embodiments, a surgeon might know a desired value for angle of inclination or angle of forward flexion well in advance of surgery, and in such a case an orientation of a mounting projection on the alignment guide can be adjusted to match the desired angle or angles. In such an embodiment, a surgeon need only match the orientation of the alignment guide during surgery without making further adjustments.

Once the alignment guide is created in software, it can be fabricated physically, as shown by step 308. While any of a variety of manufacturing processes can be utilized to create the patient-specific alignment guide, in some embodiments the alignment guide can be amenable to fabrication using additive manufacturing technologies, such as 3D printing, etc. Employing such technologies to fabricate the alignment guide can be cost effective, quick, and accurate to the 3-dimensional model. It will be appreciated that 3D printing and other rapid prototyping or additive manufacturing techniques available in the art can create parts of varying geometries using a large assortment of materials. In some embodiments, 3D printing using any of a variety of biocompatible polymers can be utilized.

Using 3D printing or some other on-demand manufacturing process, it is possible for a surgeon or other user to conduct a scan, perform 3-dimensional modeling, and fabricate an alignment guide all in an operating theater and on the same day. In many instances, however, it can be more efficient to conduct scans, modeling, and fabrication in advance of an operation and at a remote site therefrom. Accordingly, in such embodiments, a patient-specific alignment guide can be delivered to a surgeon or other user in advance of a procedure, as shown by step 310.

FIGS. 4-6 illustrate different embodiments of patient-specific alignment guides 400, 500, and 600, respectively. Each patient-specific alignment guide shares some common features, including, as mentioned above, the presence of a mounting projection 404, 504, and 604 that can be used to, for example, couple the second electronic position sensor 204 thereto during a procedure. Each alignment guide also includes at least one surface 402, 502, 602, respectively, that is configured to be substantially a negative of a portion of the patient's anatomy. Accordingly, when the alignment guides 400, 500, 600 are pressed against, for example, a patient's acetabulum, they will only mate or abut closely thereto in one orientation because the patient-specific surfaces 402, 502, 602, respectively, match only a specific portion of the patient's anatomy.

Patient-specific alignment guides can be configured to interface with, or be positioned against, different portions of a patient's anatomy, however. For example, the patient-specific alignment guides 400, 500 are both configured to extend into an acetabular socket of a patient's pelvis. The alignment guide 600, on the other hand, is configured to contact one or more portions of a patient's acetabular rim and may not necessarily extend into (or at least extend as far into) the acetabular socket as the alignment guides 400, 500.

Further, the mounting projections 404, 504, 604, respectively, can vary from one another. The mounting projections 404 and 604, for example, provide a cannula into which another shaft might be placed, while the mounting projection 504 includes a shaft extending away from the patient-specific surface 502 along a longitudinal axis L. Regardless of the particular configuration, the mounting projections 404, 504, 604, respectively, can be configured to provide for coupling the second electronic position sensor 204 to the alignment guide when it is positioned against the corresponding anatomy of the patient.

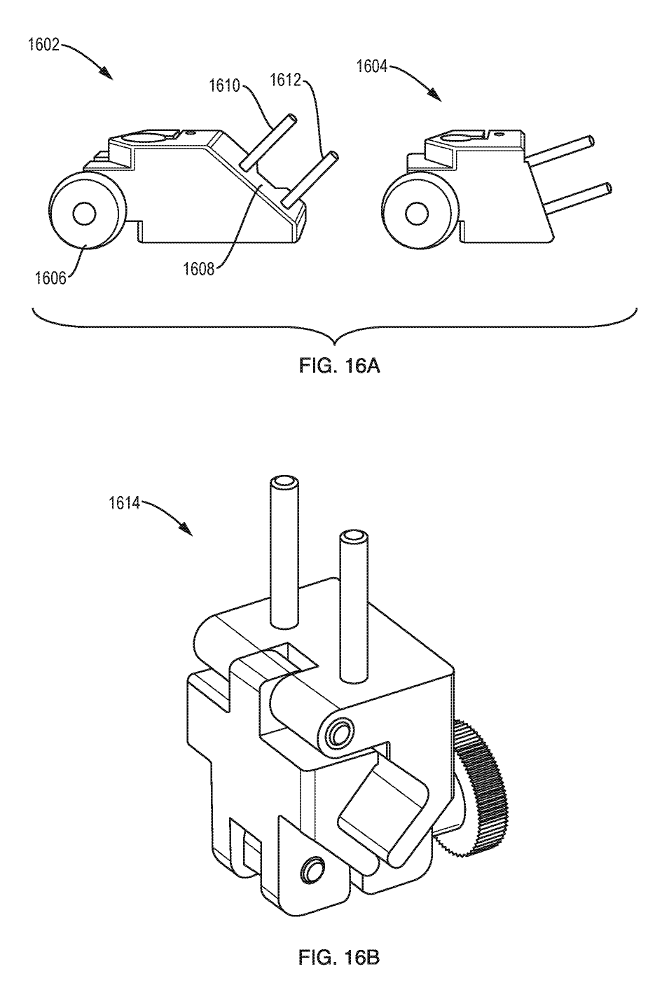

A patient-specific alignment guide can further include a mounting mechanism, such as mounting ramp 506 of the alignment guide 500. The mounting mechanism 506 can provide a mounting surface for coupling the second electronic position sensor to the patient-specific alignment guide or another instrument. The mounting mechanism 506 can, in some embodiments, be configured to position the second electronic position sensor at an angle relative to the longitudinal axis L of the mounting projections 504. In the illustrated embodiment, for example, the angle can be about 45°. Such a feature can be included because, in some embodiments, the electronic position sensors can be more accurate and/or precise when maintained in a near horizontal orientation and the angle of inclination of an acetabular implant is typically near about 45°. The mounting ramp 506, then, serves to put the second electronic position sensor in the best possible orientation during use. In some embodiments, the mounting mechanism can be a separate component selectively matable to a patient-specific alignment guide or other instrument, as shown in FIG. 16A and described in more detail below. Further, in some embodiments maintaining an electronic position sensor in a near-horizontal orientation to maximize its accuracy and/or precision may not be necessary. For example, using more advanced position sensor chips and/or software algorithms to compensate for any inaccuracies can eliminate the need for maintaining a near horizontal orientation. Accordingly, in some embodiments the mounting mechanism can orient an electronic position sensor parallel to the longitudinal axis L of the mounting projection or other instrument. One example of a separate mounting component that holds a sensor in a parallel orientation is shown in FIG. 16B.

The patient-specific alignment guide 600 illustrates another feature of some embodiments: a bore 606 for receiving a surgical pin. Inclusion of such a bore 606 can allow for the alignment guide to be temporarily secured to the acetabulum or other portion of anatomy of the patient during use. This is not necessary, as a user could simply hold the alignment guide in position, but it can provide advantages in being more steady (e.g., creating less fine movement that might throw off the measurements of the second electronic position sensor) and freeing up a surgeon or other user's hand for other tasks. It will be appreciated that the bore 606 could be positioned anywhere along the alignment guide and that more than one bore and surgical pin could be used to temporarily secure the guide to the patient's bone structure.

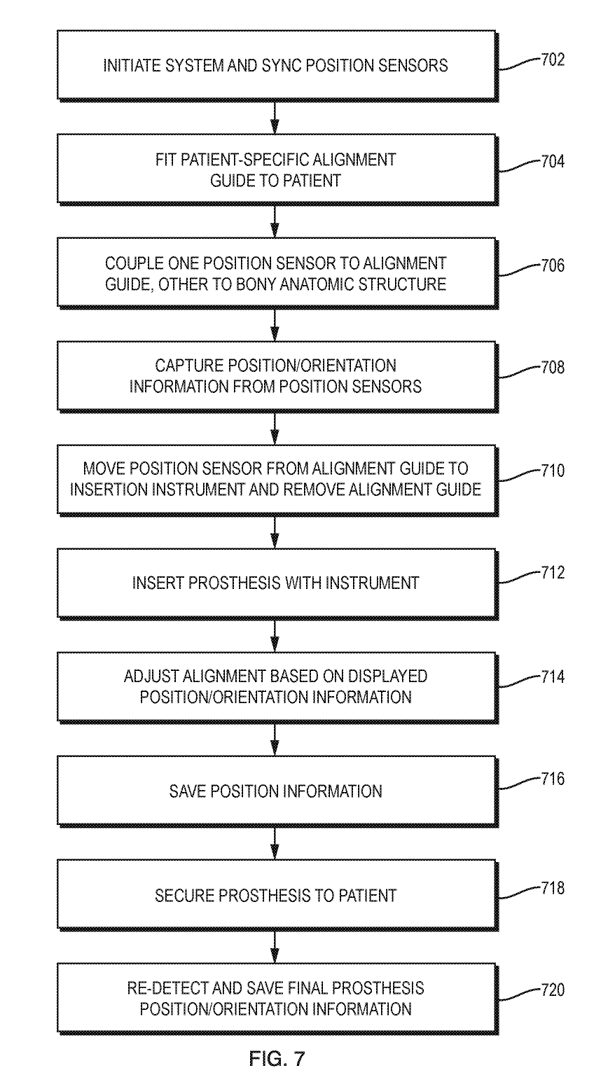

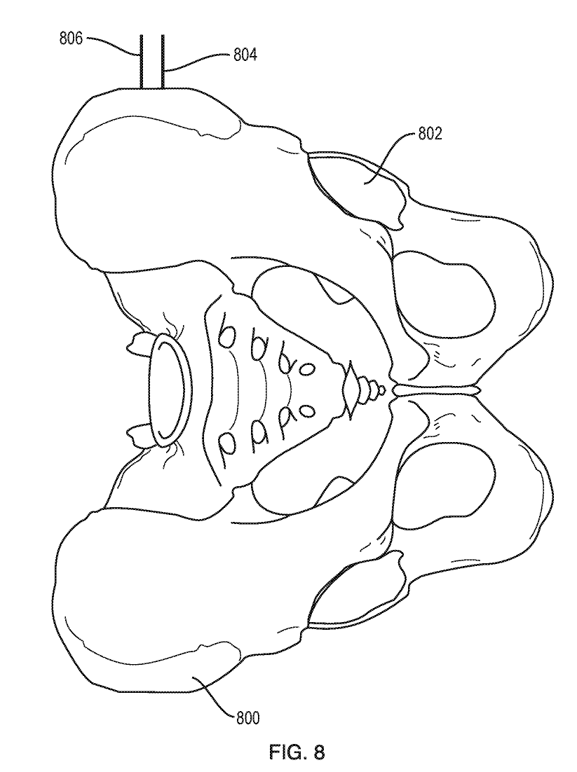

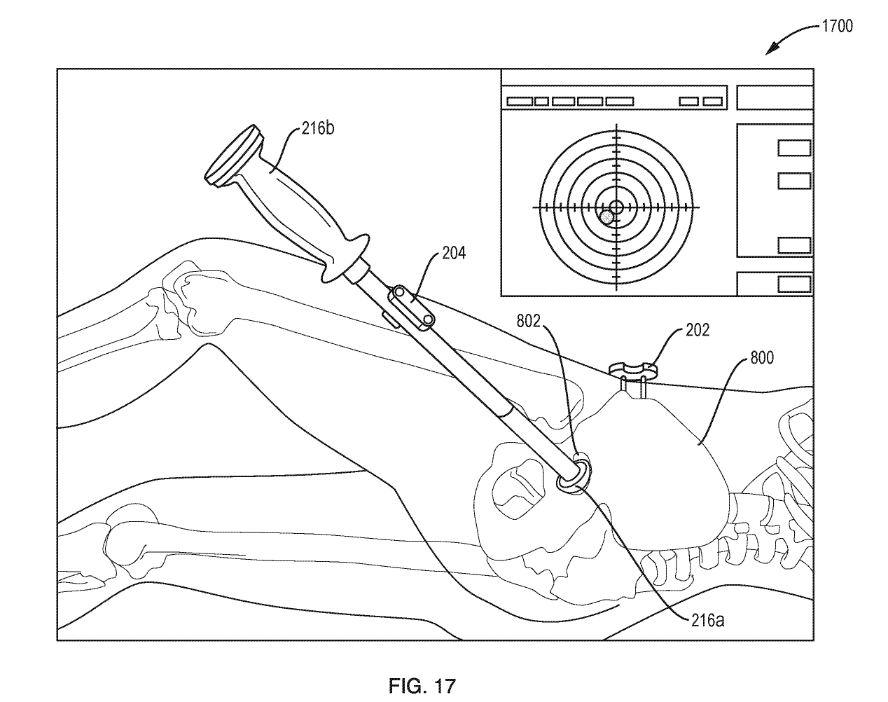

FIGS. 7-16 illustrate one embodiment of a surgical procedure according to the teachings of the present disclosure. Such a procedure is typically performed after a surgeon or other user has received a patient-specific alignment guide for use in the operation. The procedure can begin with a surgeon preparing a patient's pelvis or other bony anatomic structure to receive the first electronic position sensor 202. This can be done by driving, for example, first and second surgical pins 804, 806 into the pelvis or other bony anatomic structure 800. One advantage of the systems and methods described herein is that the pins 804, 806 can be placed anywhere on the patient's pelvis or bony anatomic structure and the location can be left to a surgeon's choice. Location of implantation can be driven, for example, by personal preference, ease of access, surgical approach, etc. For example, the illustrations of FIGS. 8, 10, and 12-15 can be a suitable configuration for a lateral approach where a patient is lying on their side. A different approach, for example the configuration shown in FIG. 17, can be a suitable configuration for an anterior approach where the patient is lying on their back. In such an orientation, the surgical pins implanted in the patient's pelvis can be rotated 90° from the orientation shown in FIGS. 8, 10, and 12-15. In the prior techniques referenced above, the pins have to be positioned precisely such that an electronic position sensor coupled thereto aligns with an anatomical plane or axis of the patient's body in a particular manner. With the systems and methods described herein, relation to the patient's anatomical planes is determined in the 3-dimensional model and is known in relation to the patient-specific alignment guide. Accordingly, the first electronic position sensor 202 need only serve as a fixed reference on the patient's pelvis or other bony anatomic structure and need not also be a direct reference to an anatomical plane or axis of the patient's body. This significantly eases use of the system, reduces complexity for a surgeon or other user, and eliminates potential sources of error in the procedure.

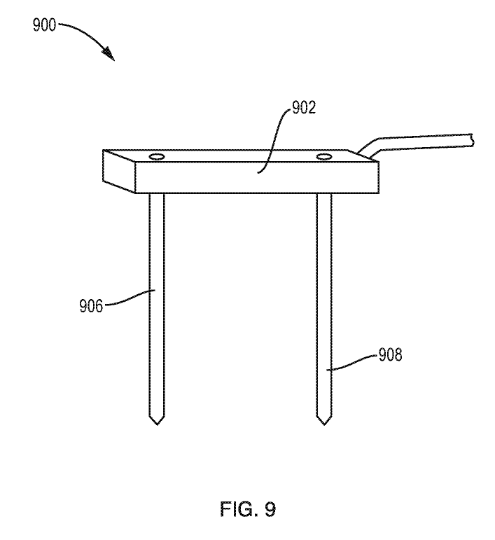

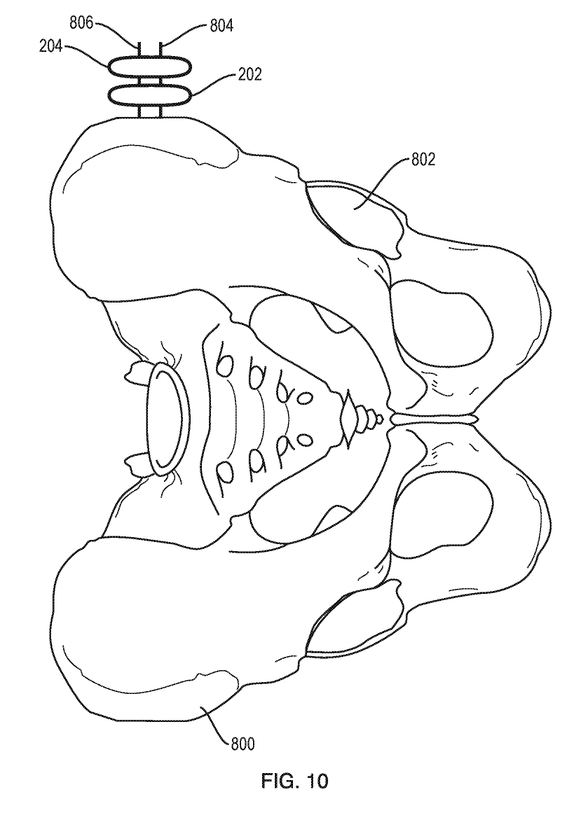

While the first electronic position sensor 202 need not be positioned at a particular place relative to the patient's pelvis or other bony anatomic structure, it can be desirable to prevent the first electronic position sensor from moving relative to the pelvis. In one embodiment, this can be accomplished by placing the pins 804 and 806 into a patient's pelvis in a parallel orientation so that they can effectively align with through-holes in an electronic position sensor and restrict movement of any electronic position sensors that may be placed thereon. To ensure the pins are positioned parallel to one another, a pin guide can be utilized. FIG. 9 illustrates one embodiment of such a pin guide 900 for placing pins on the patient's anatomy. The pin guide 900 can have a horizontal base 902 with two parallel and equal length cannulas 906, 908 extending away from the base. The cannulas 906, 908 can be arranged so that they can be used to contact a patient's bone to allow a pin to be inserted, e.g., one through each cannula, such that a sensor can be attached to the bone via the pins.

In use, a surgeon can, for example, choose to insert a first pin (e.g., pin 804) into a patient's pelvis without using the guide. In order to place the second pin (e.g., pin 806) in a parallel orientation, the pin guide 900 can be slid over the first pin such that the first pin extends through a first cannula (e.g., cannula 906). The second pin can then be driven into the patient's pelvis or other bony anatomic structure through the second, unoccupied cannula (e.g., cannula 908). The surgeon or other user can then withdraw the pin guide 900 by sliding it away from the patient's skin over the pins 804, 806, thereby leaving the pins driven into the patient's pelvis or other bony anatomic structure in a parallel orientation.

In another embodiment, a surgeon or other user can position the pin guide 900 relative to the patient's pelvis or other bony anatomic structure prior to driving either of pins 804, 806 into the bone. In such an embodiment, for example, the two cannulas 906, 908 of the pin guide 900 can be passed through two incisions (e.g., 3 mm incisions in some embodiments) made by a surgical knife into the skin over a predetermined location on the patient's pelvis or other bony anatomic structure, until a tip of both cannulas 906, 908 comes into contact with the bone. The surgical pins 804, 806 can then be passed through the cannulas 906, 908 and driven into the pelvis or other bony anatomic structure. The tips of both cannulas 906, 908 can remain in contact with the bone when the pins are being placed in the pelvis or other bony anatomic structure. The pin guide 900 can then be removed, leaving the configuration shown in FIG. 8.

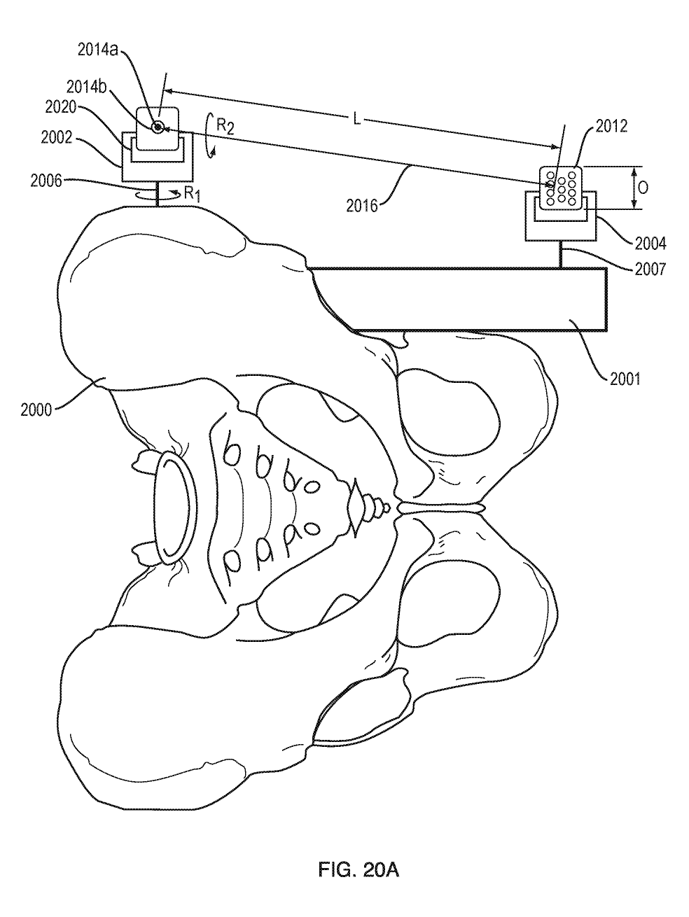

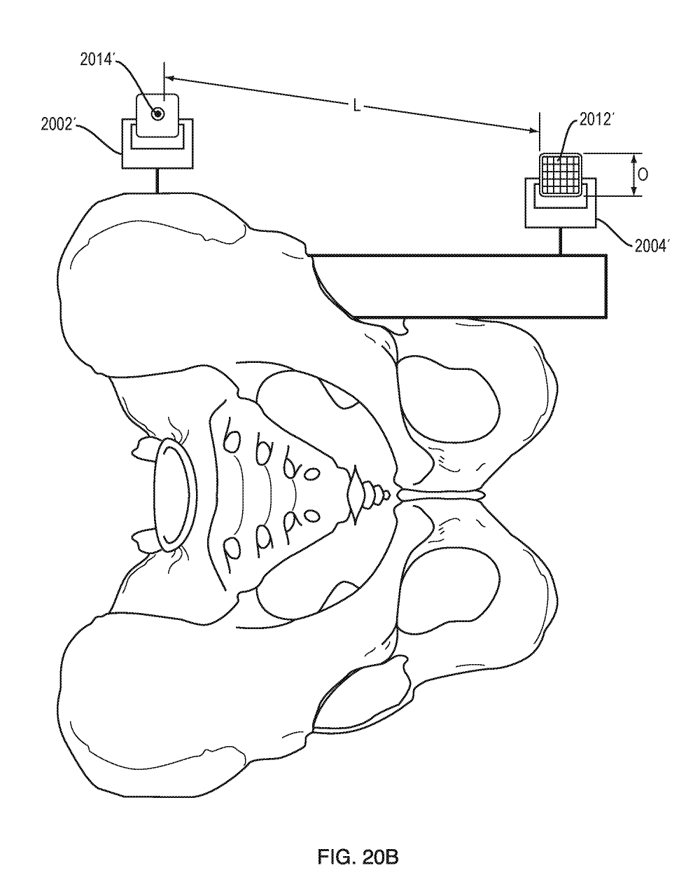

In still other embodiments, a different configuration of pins can be utilized that eliminates the need for a pin guide. For example, in some embodiments a single surgical pin can be utilized along with, for example, a coupling or other component that allows one or more electronic position sensors to be securely coupled to the surgical pin. In some embodiments, the coupling can be configured to prevent, for example, rotation of the position sensor about the pin. In other embodiments, a coupling can be utilized that permits selective adjustment of the sensor relative to the pin. One example of such an embodiment is shown in FIG. 20A, where a single pin 2006 is utilized to couple a first electronic position sensor 2002 to the pelvis or other bony anatomy 2000. The sensor 2002 can include a U-joint 2020, a ball-joint, or another mechanism that can allow adjustment of the sensor's orientation relative to the pin 2006 (e.g., rotation about the pin, sliding along a length of the pin, and pivoting about an axis transverse to the pin). Such a mechanism can also include an ability to selectively lock and prevent any and all degrees of movement once a desired position is achieved.

Having prepared the patient's pelvis or other bony anatomic structure 800 to receive one or more electronic position sensors, a surgeon or other user can proceed using the method shown in FIG. 7. The first step in such a procedure can be to initiate the system and sync the first and second electronic position sensors 202, 204, as shown at step 702. Initiating the system can include, for example, powering on the processor 206 of the tablet or other device that is running the intra-operative software. An initial interface presented to the surgeon or other user can prompt the user for entry of identifying information for the patient and/or patient-specific alignment guide. This can include, for example, entering a patient identification code, entering a code printed on the patient-specific alignment guide, scanning a barcode or other indicia accompanying the patient-specific alignment guide, etc. Upon entry of such identifying information, the intra-operative software can load information, such as the angles of inclination and forward flexion of the patient-specific alignment guide when in position against a patient's anatomy, into memory. In some embodiments, this information can be entered manually (e.g., in an embodiment where such information is printed directly on the patient-specific alignment guide rather than a patient-identifying or other code), while in other embodiments this information can be downloaded from a digital data repository via a network connection using, for example, the patient-identifying code to access the correct information.

The intra-operative software can also prompt a user to enter desired angles of inclination, forward flexion, combined anteversion, etc. if they are different than the angles of the patient-specific alignment guide when properly positioned. Entry of such information can allow the intra-operative software to perform calculations and provide guidance toward a desired position while saving a surgeon or other user from performing calculations in their head based on the angles provided by the patient-specific alignment guide.

In addition to initiating the intra-operative software, syncing of the first and second electronic position sensors 202, 204 can increase their precision, as any differences in measurement can be offset. By way of further explanation, inertial sensors of the type utilized in the first and second electronic position sensors 202, 204 can be subject to variation from one another (e.g., when oriented in the same direction, each sensor can report small differences in its orientation) and this variation can change over time (e.g., the position sensor elements can experience drift over time, typically more so with regard to motion about a vertical axis, e.g., direction, that depends on a gyroscope about a vertical axis and/or a magnetometer). Syncing the sensors can eliminate any inherent variation and can reset any drift error to zero at the commencement of a procedure.

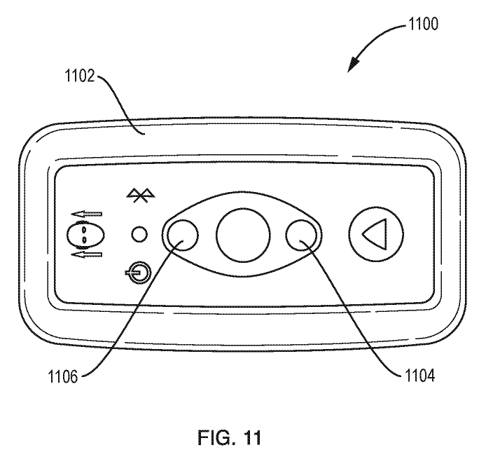

Syncing the first and second electronic position sensors 202, 204 to one another can be done in a variety of manners. For example, in some embodiments the system 200 can include a stacking tray (not shown) or other fixture to align the two sensors 202, 204 in the same orientation. Alternatively, the sensors 202, 204 can be stacked on top of one another over the pins 804, 806 that are already implanted in the patient's pelvis or other bony anatomic structure 800. Such an orientation is shown in FIG. 10. In some embodiments, stacking the sensors on the pins 804, 806 can offer the advantage of minimizing the distance traveled by the second sensor in subsequent steps (see below). This can reduce the amount of any drift or error that can be introduced by excess movement of the sensors relative to one another.

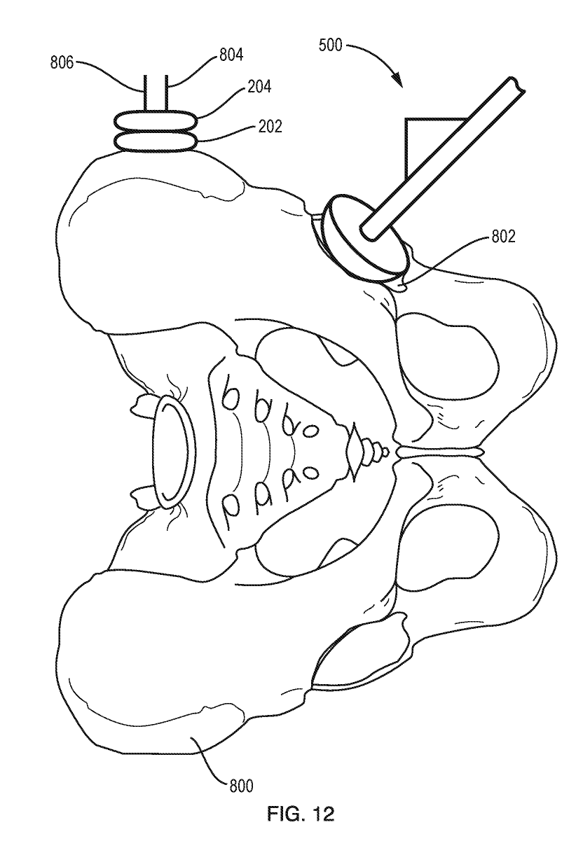

FIG. 11 illustrates one embodiment of an electronic position sensor 1100 in more detail. The sensor 1100 can include a housing/enclosure 1102 that contains the various components of the sensor, as described below. The housing/enclosure 1102 can also include a plurality of through-holes 1104, 1106 formed therein that can be configured to receive surgical pins 804, 806. The spacing of the holes 1104, 1106 and the pins 804, 806, along with the diameters thereof, can be selected such that relative movement between the sensor 1100 and the pins 804, 806 is prohibited.

The electronic position sensor units described herein can be inexpensive, highly accurate, digital components able to communicate with the intra-operative software program running on a computer processor, personal computer (PC), or hand-held electronic device (e.g., a smart phone or electronic tablet), to accurately determine the pelvic tilt and both the angles of inclination and forward flexion of the native acetabulum and prosthesis. The determination of these angles can also be seen and read by the surgeon via a portable digital visual display, thereby removing the need for a PC. In one embodiment, the measuring system can continuously monitor the patient's pelvic position and, as a consequence of this capability, the surgeon can effectively ensure an accurate angular placement of the acetabular prosthesis within the host's native bone without compromising stability of the reconstructed joint.

In some embodiments, the first electronic position sensor 202 can be a micro-electromechanical system (MEMS) multi-axis position sensor that is calibrated in all three Axes 0-2. Measuring the position in Axis 1 and Axis 2, for example, can reveal the pelvic axis and AP tilt, respectively. By way of further example, measuring position in Axis 0 can serve as a reference axis for calculating a prosthetic acetabular cup's forward flexion angle. The first electronic position sensor 202 can be coupled to the bony pelvis (or other bony anatomic structure in the case of operations on knees, wrists, shoulders, or other parts of the body) at any desired location, as described above, using, e.g., securing pins 804,806. In one embodiment the sensor 202 can wirelessly communicate with a computer processor, PC, or hand-held electronic device running the intra-operative software.

The second electronic position sensor 204 can be an electronic position and rotation sensor much like the first electronic position sensor unit, and it can also be calibrated to make digital measurements in all three Axes 0-2. The second sensor 204 can measure absolute angles for determining the position of the acetabular prosthetic implant as a single electronic calculation. True angles of inclination and forward flexion can be calculated by the intra-operative software when the second sensor 204 is used in combination with the first sensor 202. In one embodiment, the second sensor 204 can communicate wirelessly with the computer processor, PC, or hand-held electronic device running the intra-operative software. In one embodiment, the second sensor 204 can communicate wirelessly with the first sensor 202.

As described below via the exemplary hip joint prosthetic surgery, the second sensor unit 204 can be primarily focused upon determining the position of the patient-specific alignment guide and the acetabular prosthesis when each one is being implanted in situ. To achieve this, the second sensor unit 204 can be typically coupled to the patient-specific alignment guide or the cup impactor or other insertion instrument in order to show the true position and placement orientation for the implanted prosthesis at that moment in time.

Accordingly, the second sensor unit 204 can measure the absolute angles for both of two different parameters: (i) the absolute angles then existing for the host's acetabulum (i.e., the patient's hip socket); and (ii) the absolute angles of the acetabular prosthesis then being implanted by the surgeon.

The electronic position sensors 202, 204 used herein can have at least one orientation sensor and at least one transmitter, or wireless antenna. The transmitter can be any of a variety of types used to transmit information, including wirelessly, to a computer or tablet. In one embodiment, each of the first and second electronic position sensors 202, 204 can include a BLUETOOTH transceiver. The orientation sensors can preferably specify the tilt of the sensor with respect to orthogonal axes (such as x-y-z axes) and heading with respect to an external field. The external field measured by the first and second electronic position sensors 202, 204 can be the Earth's magnetic field in some embodiments.

By way of further example, an electronic position sensor described herein can include the following components: a tilt sensor module and a direction sensor module built in a MEMS (micro electro mechanical system) chip; a Bluetooth module for wireless communication; a micro controller unit to operate the systems; an internal power source; and a printed circuit board onto which the other components can be placed.

In exemplary embodiments, the tilt sensor can be an accelerometer capable of measuring degrees of tilt from the true horizontal plane in three different axes. It can be used for sensing position and degree of tilt of a patient's pelvis from a vertical position. It can also be used for sensing a degree of tilt of an implant from a horizontal plane.

The direction sensor can be a digital magnetometer capable of showing the direction of the axis of an object. The sensor can be used to sense the direction of the pelvis. An additional sensor can sense the implant vector of the acetabular cup when attached to an instrument that is used for placement of the cup.

An exemplary device can include a 3D digital linear acceleration sensor, a 3D digital gyroscope, and a 3D digital magnetic sensor. In some embodiments, operation can be conducted without use of a magnetic sensor to avoid interference from metal objects in an operating room, etc. The output from such a system can be converted in software, firmware, or the like into the tilt data utilized by the systems and methods described herein.

In some embodiments, the devices can additionally include a feature to monitor any changes in length of the operative leg 2001, as shown in FIG. 20A. For example, a sensor can be utilized to assist the surgeon in measuring any difference in the operative leg length during surgery to aid in correcting any pre-existing leg length inequality and/or to avoid any undesirable post-operative leg length change or discrepancy. In one exemplary embodiment, the first electronic position sensor 2002, which can be fixed to a patient's pelvis as described above, can include a leg length sensor configured to detect a distance between the first sensor 2002 and a second electronic position sensor 2004 that can be coupled to a patient's femur (e.g., the greater trochanter of the femur in some embodiments) prior to dislocation or following prosthesis implantation. A variety of leg length measurement sensors can be employed, including sensors utilizing lasers, ultrasound, radio frequency or infrared signals, magnetic fields, and physical components such as cable position transducers and linear encoders. In some embodiments, a laser-based leg length measurement sensor can be employed. Such a sensor can include, for example, a laser emitter 2014a and a receiver 2014b. The laser emitter 2014a can be aimed such that the beam 2016 reflects off the second sensor 2004 and returns to the receiver 2014b. Because a distance between the laser emitter 2014a and the receiver 2014b of the first sensor 2002 can be fixed, trigonometry can be used to calculate the length L between the first electronic position sensor 2002 to the second electronic position sensor 2004 based on where in a focal plane of the receiver 2014b the reflected laser beam 2016 is detected.

The first electronic position sensor 2002 and second electronic position sensor 2004 can additionally be configured to detect femoral offset both before dislocation of the femur and following prosthesis implantation. In one embodiment, the first position sensor 2002 can include a laser emitter or other light source, which can in some cases be separate from the above-mentioned laser emitter 2014a utilized for a leg length measurement sensor. The second electronic position sensor 2004 can include a photo receptor array 2012 that can detect the laser beam emitted from the first electronic position sensor 2002. The photo receptor array 2012 can be a two-dimensional array of photo receptors that are individually able to detect an output from the laser. The particular receptor that detects the laser can be recorded in a memory and changes in which receptor detects the laser before and after a procedure can be utilized to compute changes in femoral offset. In some embodiments, faces 2002a, 2004a of the first and second electronic position sensors 2002, 2004 can be oriented such that they are parallel to one another to ensure that any laser beam 2016 or other signal or measuring device is properly aligned to calculate the length L and/or offset O shown in FIG. 20A. To this end, one or more of the first and second sensors 2002, 2004 can include a U-joint 2020 that permits the sensor to pivot about two axes, illustrated with reference to sensor 2002 as R₁ and R₂.

As noted above, a baseline length L can be measured pre-operatively (e.g., prior to dislocation of the femur) and repeatedly during the operation (e.g., following re-approximation of the femur to the pelvis via trial or final prosthetic cup and/or femur components) to ensure that there is no post-operative leg length discrepancy (or that there is a desired change in leg length measurement). Femoral offset O can be determined along with leg length L or separately therefrom based upon which receptor in the photo receptor array 2012 is activated by a laser emitted from the first sensor 2002. A processor can translate changes in detection of the laser within in the 2D photoreceptor array 2012 into a measurement of femoral offset change. As with leg length measurement, a baseline offset can be measured pre-operatively intra-operatively to calculate any change in femoral offset that resulted from the procedure. In one embodiment, a pre-operative leg length and offset measurement can be taken prior to initiating the operative method shown in FIG. 7. For example, one or more surgical pins or another mounting mechanism can be placed in each of the patient's pelvis and femur to provide a fixed point for coupling the first and second sensors 2002, 2004. Leg length and offset measurements can be taken as described above. The sensors 2002, 2004 can then be removed from the pins, the femur can be dislocated from the pelvis and prepared to accept the femur prosthesis, and the procedure of FIG. 7 can be performed on the exposed acetabular cup of the pelvis. Note that the pins can remain in the patient following implantation until the end of the procedure such that the same sensor positioning relative to each part of anatomy can be utilized for subsequent measurements.

In an alternative embodiment, illustrated in FIG. 20B, a second electronic position sensor 2004′ can include a visual guide 2012′ in the form of a printed or engraved grid for a visual determination of the femoral offset O rather than a computed determination based on the photoreceptor array 2012. For example, a visible light targeting laser 2014′ can be emitted from the first sensor 2002′ and the laser beam can be observed on the visual guide 2012′. The operator can then read off (if the visual guide 2012′ is so marked) or compute offset measurements based on the position of the targeting laser on the visual guide. Such an embodiment can provide an advantage by allowing the elimination of the photoreceptor array 2012 described above, which can allow the second position sensor 2004′ to be smaller and require less energy to operate. It is also possible in certain embodiments that the visual guide 2012′ contains no marking at all, e.g., a plain surface, and a user can mark the surface with a marker or other instrument to note offset measurements. Such markings could, of course, also be made on the visual guide 2012′.

In still other embodiments, the sensors can be configured to measure leg length L without including femoral offset. In such an embodiment, the second sensor 2004 can have a reduced size, as there need not be a photoreceptor array 2012 or surface including a visual guide 2012′. The second sensor 2004 can instead serve simply as a reflective target for the laser rangefinder of the first sensor 2002 described above. As described herein, the second sensor 2002 in such embodiments can include a MEMS position-detecting sensor for use in embodiments where dual sensors are employed, or it can be nothing more than a reflective target for use in embodiments where a single sensor is employed.

In some embodiments, a Bluetooth module can be included in an electronic position sensor and utilized to provide a wireless mode of communication between the sensor and the central processor unit (such as a PC, tablet, etc.) running the intra-operative software. It can wirelessly transfer raw data from the sensor to the digital data processor where the intra-operative software can receive the data and calculate the position angles of the pelvis and implant. A graphical user interface can be provided to show the data to a user.

In an alternative embodiment, the two electronic position sensors can be wired together. The use of a wired connection can advantageously allow for a constant connection that is less susceptible to interference and other transmission errors. Further, the removal of the Bluetooth or other wireless communication modules can reduce the overall size of the electronic position sensors. Moreover, in some embodiments the second electronic position sensor may not require a battery to support a wireless connection, thus the overall size of the second electronic position sensor can be further reduced. In still other embodiments, both electronic position sensors can be wired to one another, and one or more of the electronic position sensors can be wired to the digital data processor. In embodiments where all of the components are wired together in this manner, Bluetooth or other wireless communication modules can be removed from both of the electronic position sensors. Further, if the wired connection supports both data and power, the electronic position sensors can operate without the need for batteries, which can allow the size of both components to be reduced.

A microcontroller unit can manage all of functions and performance of the main electronic components of the sensor.

The power source for the electronic position sensor can be any of a variety of batteries that can provide sufficient power to the sensor. Given the demands of sensing and transmitting data in order to provide a surgeon with real time information, for example, on the tilt of a patient's pelvis, batteries with high energy density and/or capacity, such as those that are sometimes used in products such as cameras and toys, can be preferred over batteries with lower energy density and/or capacity. In addition, in order to achieve battery life that is sufficient for the sensor to be used throughout a surgery, the firmware of the Bluetooth module can be adjusted to optimize its energy use and an oscillator can be added to aid in controlling power consumption. For example, a typical Bluetooth transceiver power consumption profile can include an initial spike followed by a lower plateau. Accordingly, optimization can be performed to determine if the most efficient mode of operation is continuous transmission (i.e., operating at the “plateau” of the profile) or repeated transmission and hibernation cycles. In still other embodiments, such modifications can be avoided in favor or using, for example, low energy protocols like Bluetooth LE. Further still, in embodiments where wired connections are used, batteries may not be needed and a dedicated power source can be provided. Such a dedicated power source can be a computer or a direct connection to a power source.

Returning to the surgical method illustrated in FIG. 7, at step 704 a surgeon or other user can fit a patent-specific alignment guide to a patient such that a patient-specific surface thereof interfaces with the portion of the patient's anatomy that it mirrors. FIG. 12 illustrates the patient-specific alignment guide 500 being positioned within the acetabulum 802 of the patient's pelvis 800. Note that, at this point, the first and second electronic position sensors 202, 204 can remain stacked on the surgical pins 804, 806 or can be placed in a holding tray or other receptacle away from the patient. Note also that the syncing process described above and shown at step 702 of FIG. 7 can be performed after positioning the patient-specific alignment guide in some embodiments. Moreover, in some embodiments one or more surgical pins can be driven into the acetabulum, acetabular rim, or periacetabular area surrounding the acetabulum to secure the alignment guide 500 during use.

At step 706 of FIG. 7, a surgeon or other user can move the first electronic position sensor 202 to the surgical pins 804, 806—if it was not already positioned there—and can move the second electronic position sensor 204 to the patient-specific alignment guide 500. This can be done, for example, by placing the through holes of the second electronic position sensor 204 over two pins (not shown) projecting from the mounting mechanism 506.

During this movement of the second electronic position sensor 204, position and angle data can be transmitted to the processor 206 to track the movement of the second electronic position sensor 204. Alternatively, in some embodiments data transfer during the move may not take place and the sensor can transmit data regarding changes in its position and/or orientation only after the move is complete. Detection of a complete movement can be done automatically, for example, by detecting a rate of change in angular or positional measures, or manually, for example by a user pressing a button on the display/interface 212.

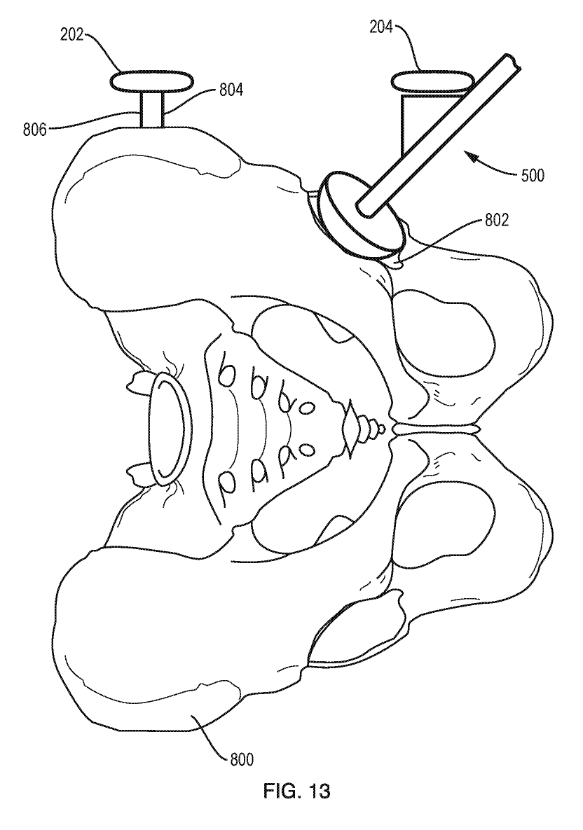

Regardless of how the motion capture is initiated or otherwise implemented, the processor can receive position and orientation data from the first and second electronic position sensors 202, 204 when in the configuration shown in FIG. 13, as shown in step 708 of FIG. 7. This can serve as a reference position that can be returned to after the patient-specific alignment guide 500 is removed and an acetabular implant is inserted in its place. Moreover, because the relative angles between the alignment guide 500 and the anatomical planes/axes of the patient's body are known when the patient-specific alignment guide is in position, the reference positions of the first and second electronic sensors 202, 204 can be related to angles relative to the anatomical planes/axes of the patient's body through calculations performed by the processor 206. This can allow the processor 206 to guide a surgeon or other user, via, for example, data shown on the display/interface 212, to an implant orientation having a desired angle of inclination and/or forward flexion.

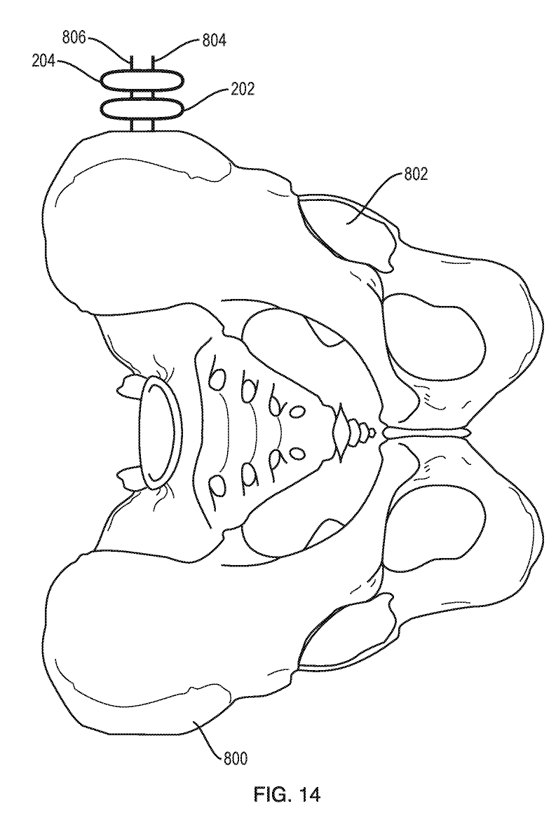

At step 710 of FIG. 7, the first and second electronic positions sensors 202, 204 can be moved back to a standby position, e.g., where both sensors are stacked on the pins 804, 806 or on a fixture remote from the patient, and the patient-specific alignment guide 500 can be removed from the acetabulum 802 of the pelvis 800. Such an arrangement is shown in FIG. 14. At this point, any necessary preparation for an implant, e.g., reaming, etc., can be performed. An impactor 216b or other insertion instrument can be coupled to an acetabular cup implant 216a or other prosthesis and readied for insertion into the acetabulum 802. This process can include coupling the second position sensor 204 to the impactor 216b such that the second position sensor can transmit data on a position of the impactor 216b and the implant 216a coupled thereto.

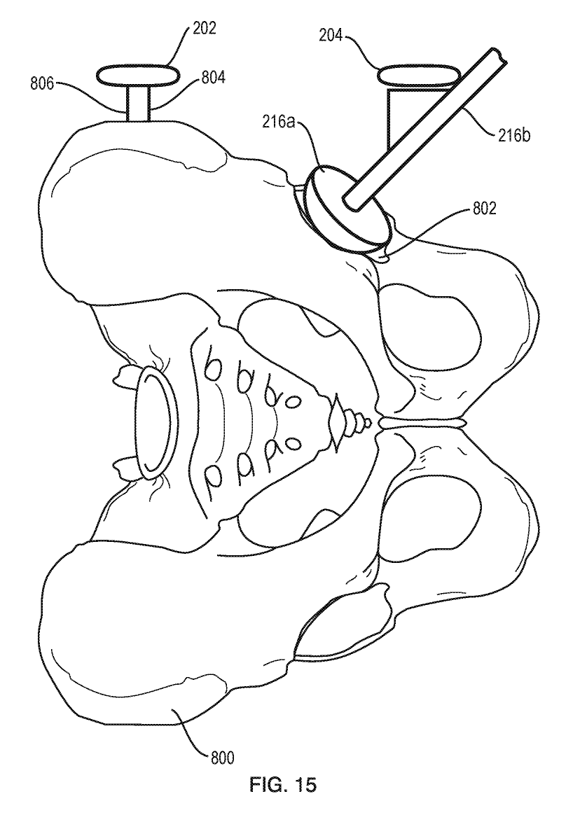

At step 712 of FIG. 7, and as shown in FIG. 15, the prosthesis 216a can be inserted into the acetabulum 802 of the patient's pelvis 800 using the impactor 216b. The first and second electronic position sensors 202, 204 can transmit position and orientation data to the processor 206, which can calculate the current, real-time position and/or orientation of the patient's pelvis or the prosthesis/impactor, as well as provide real-time updates to a surgeon or other user on any changes in position and orientation thereof. Using calculations performed by the processor 206, such position and/or orientation information can be displayed in relation to the bony anatomic structure (i.e., the pelvis) or any desired planes, including, for example, the sagittal, coronal, or transverse planes, as well as any other planes desired by a user, such as the anterior pelvic plane discussed above.

Note that FIG. 15 illustrates that the second electronic position sensor 204 oriented relative to the impactor 216b in a similar manner as it was oriented relative to the mounting projection of the patient-specific alignment guide 500, i.e., at an angle that maintains the sensor 204 closer to a horizontal orientation where its precision and/or accuracy are improved. As explained above, however, in some embodiments such an orientation need not be maintained due to the use of improved electronic position sensors that have greater accuracy and/or precision, or due to the implementation of algorithms that compensate for any error introduced at severe positioning angles.

In order to couple the sensor 204 to a device that may not be intended for use with such a sensor, a mounting adapter 1602 can be included. As shown in FIGS. 16A and 16B, the mounting adapter 1602 (or 1604 or 1614, which have different angles of orientation) can be configured to couple to any generally elongate impactor or other instrument using a compression fit controlled by thumb-knob 1606. The adapter 1602 can include a mounting surface 1608 extending at an angle from the through-hole that receives an instrument shaft. The mounting surface can provide an attachment position, as well as a locking mechanism, to couple the second electronic position sensor to the implant placement instrument, e.g., cup impactor. In some embodiments, a plurality of pins 1610, 1612 can extend from the mounting surface 1608. The pins 1610, 1612 can be spaced and sized similar to the pins 804, 806 so as to receive the second electronic position sensor 204 thereon and prevent relative movement between the sensor 204 and the adapter 1602. The mounting surface 1608 can extend at any angle from the through-hole, including orientations in which the mounting surface is parallel to the through-hole (e.g., see adapter 1614 of FIG. 16B), perpendicular thereto, and extending at a 45° angle relative thereto (e.g., see adapter 1602 of FIG. 16A), as well as any intermediate angle.

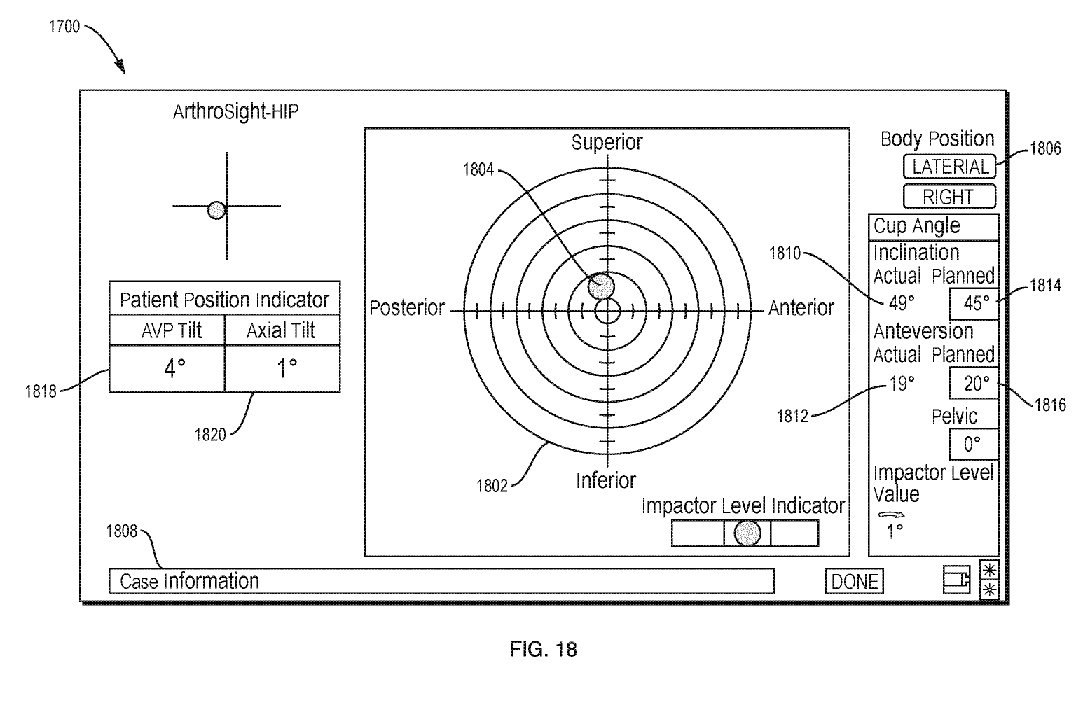

In the next illustrated step of FIG. 7, step 714, a surgeon or other user adjusts alignment of a prosthesis based on feedback provided by the display/interface 212 to arrive at a desired angle of inclination and/or angle of forward flexion and/or angle of combined anteversion for the prosthesis. FIGS. 17 and 18 illustrate one embodiment of this process in more detail, where an exemplary display 1700 is shown with a “target” graphic to help guide a surgeon or other user in positioning the acetabular cup implant 216a into a desired position using the impactor 216b. The graphic, including current and desired angular measurements along a right side thereof in some embodiments, can be calculated and updated in real time based on data received from the first position sensor 202 that is coupled to the patient's pelvis and the second position sensor 204 that is coupled to the impactor. Note that in the illustration of FIG. 17, the second sensor 204 is shown coupled to the impactor 216b in an orientation parallel to a longitudinal axis thereof, as described above.

With reference to the detail view of display 1700 shown in FIG. 18, the “target” guidance graphic 1802 can be centrally located to provide easily-ascertainable visual guidance to a surgeon or other user regarding positioning of the prosthesis. The graphic can include color coding in some embodiments, such that the positioning dot 1804 symbolizing the position/orientation of the prosthesis can be colored, e.g., green if within 5° (or less) of desired or planned angle, yellow if within 5-10°, and red if farther than 10° from the desired or planned angle.

On the right hand side of the display 1700 can be indications 1806 of surgical approach and operative side (e.g., right or left hip, etc.) or other identifying information, such as a patient ID, patient-specific alignment guide ID, prosthesis ID, etc. Further information can also be included along a bottom portion 1808 of the display 1700. The display can also include a portion showing numeric prosthesis position and orientation information. Example measures can include actual angle of inclination 1810 and actual angle of forward flexion/anteversion 1812, as well planned angles of inclination 1814 and forward flexion 1816. The latter angles can be adjustable by a user via an interface (e.g., a touchscreen of a tablet device, etc.).

Further, the display 1700 can include a portion showing anterior/posterior tilt 1818 and axial tilt 1820 of the pelvis. This can be computed, for example, based on data captured from the first electronic position sensor 202 and the known orientation of the second electronic position sensor 204 when coupled to the patient-specific alignment guide. The result is a display that shows a surgeon or other user the orientation of the prosthesis, the orientation of the patient's pelvis, and guidance toward a desired position for the prosthesis.

Once a desired position and orientation has been achieved, a surgeon or other user can fix the implant in place using a conventional method. As noted in step 716 of FIG. 7, after fixation a surgeon can elect to save the final positioning information of the prosthesis. In some embodiments, however, additional fixation elements, such as screws, cement, or other methods known in the art can be utilized to secure the prosthesis to the patient, as shown by step 718 in FIG. 7. Typically, the impactor 216b or other insertion instrument is removed from the prosthesis to facilitate application of any fixation method. Such installation can cause movement of the implant in some cases, so a surgeon or other user might also elect to re-attach the impactor 216b or other insertion instrument after installing any screws or other fixation elements to measure and save an ultimate final position and/or orientation of the prosthesis, as shown in step 720 of FIG. 7.

The above exemplary description provided patient-specific information via the use of a 3D scan of a patient's anatomy and subsequent creation of a patient-specific alignment guide that could be used to determine a reference position during a surgical procedure. In another embodiment, intra-operative x-ray or scanning of a patient can be utilized to produce patient-specific reference alignment information during a surgery without the use of a separate patient-specific alignment guide.

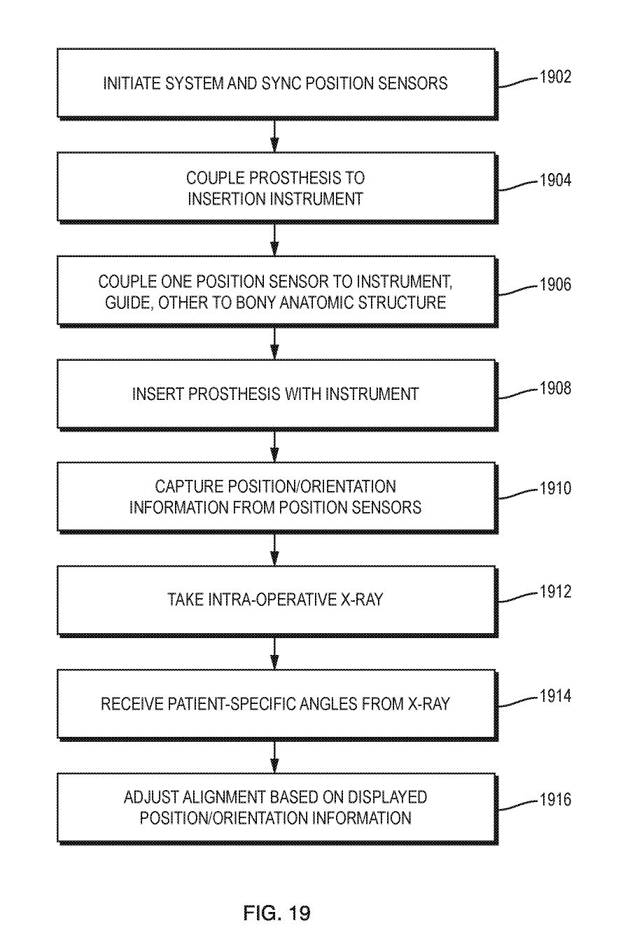

FIG. 19 illustrates one embodiment of such a method. The method can parallel the method shown in FIG. 7 for several of the steps, including the system initiation and electronic position sensor syncing process of step 1902. Rather than proceeding to use a patient-specific alignment guide, however, the method can include coupling a prosthesis to an insertion instrument (step 1904), coupling one position sensor to the insertion instrument and another to the patient's bony anatomic structure (step 1906), inserting the prosthesis with the instrument (step 1908), and capturing position and orientation information from the position sensors (step 1910).

Performing these steps can provide relative position and orientation information between the two electronic position sensors, but does not provide a reference to a position and orientation specific to the patient's anatomy. In order to determine this reference, one or more intra-operative x-ray images can be taken while the prosthesis is positioned against or within the bony anatomic structure (e.g., acetabulum of the patient's pelvis), as shown in step 1912. Using these images, absolute angles of inclination, forward flexion, etc. can be measured for the current position of the prosthesis. An example of software that could be adapted to perform such measurements is provided in U.S. patent application Ser. No. 13/187,916, filed Jul. 21, 2011, entitled “INDEPENDENT DIGITAL TEMPLATING SOFTWARE, AND METHODS AND SYSTEMS FOR USING SAME” (atty. docket no. 112428-2), the entire contents of which are incorporated herein by reference.

The measured angles can be entered into or otherwise communicated to the intra-operative software, as shown in step 1914. The intra-operative software can track further movement of the implant and provide calculated position and orientation information, such as angle of inclination, forward flexion, combined anteversion, etc. This can permit a user to adjust the positioning and/or alignment of the prosthesis based on information displayed by the intra-operative software, as shown in step 1916. Once a desired position and orientation are achieved, the prosthesis can be implanted, secured, and final positioning information can be saved in a similar manner as described above.

Though the above-described method includes collecting information from two electronic position sensors—one coupled to an implantation instrument and the other coupled to a patient's pelvis or other bony anatomy—in another embodiment the reference position established by the intra-operative x-ray images can be utilized in connection with a single electronic position sensor coupled to the implantation instrument. That is, information received from the single electronic position sensor can be used to track movement relative to the reference position established from the intra-operative x-ray images. This method of operation operates under an assumption that a patient's pelvis or other bony anatomy does not move during the procedure, as any movement thereof would not be tracked due to the absence of a second electronic position sensor coupled thereto. Nonetheless, this method of operation can still provide increased accuracy and precision in placing a prosthesis relative to some prior techniques, e.g., those that rely purely on surgeon estimation, etc.

Various alternative embodiments are also possible. For example, as shown in FIG. 20A, each of first and second electronic position sensors 2002, 2004 can be mounted to a patient's pelvis 2000 or femur 2001 using a single pin 2006, 2007, respectively. Each of the electronic position sensors 2002, 2004 can have a U-joint 2020 that permits the sensor to be pivoted about two axes, illustrated with reference to sensor 2002 as R₁ and R₂. The first and second electronic position sensors 2002, 2004 can each have a height and a width in the range of approximately 15.0 mm to approximately 35.0 mm. In one embodiment, each of the first and second electronic position sensors 2002, 2004 can have a height and a width of approximately 25.0 mm. The first and second electronic position sensors 2002, 2004 can employ various sensors to measure both the pre- and intra-operative leg lengths L and femoral offsets O. The pre-operative measurement of leg length and femoral offset can be performed, for example, prior to the steps shown in FIG. 7 before the femur is dislocated from the patient's pelvis. Thus, a reference measurement can be saved to establish a basis for comparison intra-operatively as trial and/or final prosthetic components are fitted to the patient.

As noted above, in some embodiments laser-based sensors can be utilized to perform one or more of leg length measurement and femoral offset measurement. A variety of different configurations are possible. For example, the first electronic position sensor 2002 can include a laser emitter 2014a and a receiver 2014b to conduct leg length measurements. The laser emitter 2014a can, in some embodiments, be an invisible light beam and, in such embodiments, a further visible light targeting laser (not shown) can be included in the first sensor 2002 to aid in positioning the first and second sensors 2002, 2004 relative to one another. In some embodiments, the second electronic position sensor 2004 can include a receiver 2012 configured to detect a beam 2016 from the laser emitter 2014a or a visible targeting laser to determine femoral offset, as described above. In other embodiments, the arrangement of these and other components can be changed such that, for example, the second electronic position sensor 2004 can include the one or more laser emitters and the first electronic position sensor 2002 can include a receiver. In addition, the positions of the first and second position sensors 2002, 2004 can be reversed, such that the first sensor 2002 can be coupled to the patient's femur and the second sensor 2004 can be coupled to the patient's pelvis. The receiver 2012 can include a two-dimensional array of photo receptors. Any number of photo receptors can be included. In use, the first electronic position sensor 2002 can be aimed at the second electronic sensor 2004 such that respective faces 2002a, 2004a, of the first and second electronic sensors 2002, 2004 are parallel to one another. The laser emitter 2014a on the first electronic position sensor 2002 can be activated and directed at the face 2004a of the second position sensor 2004. The light beam 2016 can reflect off the face 2004a of the second position sensor 2004 and can be detected by the receiver 2014b of the first electronic position sensor. A processor in communication with the first electronic position sensor 2002 can calculate the distance L from the first electronic position sensor 2002 to the second electronic position sensor 2004. Further, the laser light 2016 can be detected by the receptors in the photo receptor array 2012 and, based on which receptor in the array detects the light, the processor can calculate a femoral offset measurement O. In an embodiment where the laser emitter 2014a emits invisible light, a separate visible light targeting beam can be employed to help a user orient the two sensors 2002, 2004 and, in some cases, to activate the photoreceptors of the receiver array 2012.

A first measurement of leg length and femoral offset can be taken prior to dislocation of the femur after one or more surgical pins, such as pins 2006, 2007, are secured to the patient's pelvis (e.g., near the iliac crest of the pelvis) and to the patient's femur (e.g., in the greater trochanter of the femur). The operation can then proceed as described above and shown in FIG. 7. Additional measurements of leg length and femoral offset can be recorded in connection with steps 714 and 720 to ensure that the prosthesis is properly aligned before ending a procedure.

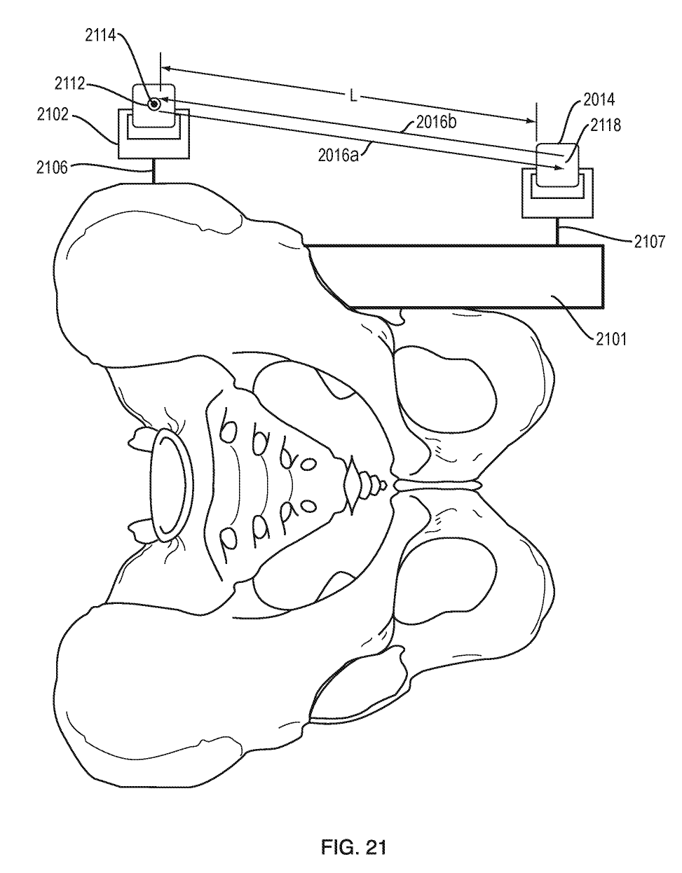

In a further alternative embodiment shown in FIG. 21, one active electronic position sensor 2102 can be used in conjunction with a reflector 2104. The electronic position sensor 2102 can be substantially similar to the first electronic sensor 2002, including a laser emitter 2114a and a receiver 2112 to measure leg length of a patient. The reflector 2104 can have a surface 2118 capable of reflecting an incoming laser signal 2116a back to the electronic position sensor 2102 (the reflected beam is shown as 2116b), but otherwise can contain no other electronic components. In such an embodiment, the reflector 2104 can be placed on the patient's femur 2101 (e.g., on the greater trochanter) using a pin 2107 to permit the system to determine an initial, pre-operative, leg length measurement. The laser emitter 2114 on the electronic position sensor 2102 can be activated and directed at the reflector 2104. The laser beam 2116a can be reflected off the face 2118 of the reflector 2104 and received back at the electronic position sensor 2102 by the receiver 2112 (as shown by beam 2116b). Based on the location of the reflected beam 2116b in a focal plane of the receiver 2112 and a known distance between the laser emitter 2114 and the receiver 2112, a distance L between the electronic position sensor 2102 and the reflector 2104 can be calculated. Following leg length measurement, the electronic position sensor 2102 can be repeatedly swapped between the pin 2106 and a patient-specific alignment guide or prosthesis positioning instrument to track positioning of a hip prosthesis in a manner similar to that described above.

In use, information from the single electronic position sensor 2102 can be utilized to track movement of the patient and/or an instrument, prosthesis, or other tool relative to the patient and a reference position established from docking the single electronic position sensor 2102 to a patient-specific alignment guide that interfaces with the patient's anatomy in a known manner. In such embodiments, this method of operation operates under an assumption that a patient's pelvis or other bony anatomy does not move during the time that the single electronic position sensor is moved from the patient-specific alignment guide to one of the prosthesis or instrument, as any movement thereof would not be tracked due to the absence of a second electronic position sensor coupled thereto. Nonetheless, this method of operation can still provide increased accuracy and precision in placing a prosthesis relative to some prior techniques, e.g., those that rely purely on surgeon estimation, etc. Moreover, in other embodiments the method can include repeatedly transferring the single sensor between a configuration in which it is coupled to the patient's pelvis and a configuration in which it is coupled to an instrument or implant, and movement of each of the pelvis and instrument over time can be tracked. For example, movement of the pelvis can be tracked over time by a position change between a first time and a second time that the single sensor was coupled to the pelvis in a same location (e.g., by coupling to one or more pins, as explained above).

Although this disclosure includes reference to specific embodiments, it should be understood that numerous changes may be made within the spirit and scope of the concepts described herein. Accordingly, it is intended that this disclosure not be limited to the described embodiments, but that it have the full scope defined by the language of the following claims or those added to non-provisional applications claiming priority hereto.

What is claimed is:

1. A system for use in implanting a prosthesis, comprising:
a digital data processor;
a display;
a first electronic position sensor capable of reporting information about its position and orientation in 3-dimensional space to the digital data processor;
a second electronic position sensor capable of reporting information about its position and orientation in 3-dimensional space to the digital data processor;
a patient-specific alignment guide configured to interface with a bony anatomic structure of a patient, wherein the alignment guide includes a surface created from a scan of the patient's bony anatomic structure that is substantially a negative of at least a portion of the patient's bony anatomic structure; and
application software configured to (i) receive information from the first electronic position sensor, (ii) receive information from the second electronic position sensor, and (iii) calculate and display angular relationships derived from the information received from the first and second electronic position sensors.

2. The system of claim 1, wherein the first electronic position sensor is configured to be coupled to the patient's bony anatomic structure and the second electronic position sensor is configured to be coupled to the patient-specific alignment guide.

3. The system of claim 1, wherein at least one of the first electronic position sensor and the second electronic position sensor is configured to communicate wirelessly with the digital data processor.

4. The system of claim 1,
wherein the first electronic position sensor and the second electronic position sensor are wired together; and
wherein the second electronic position sensor is configured to communicate with the first electronic position sensor through the wire.

5. The system of claim 1, wherein the bony anatomic structure is a pelvis.

6. The system of claim 5, wherein the angular relationships include any of (pelvic) axial tilt, (pelvic) anterior-posterior (AP) tilt, absolute angle of inclination, absolute angle of forward flexion, true angle of inclination, and true angle of forward flexion.

7. The system of claim 1, wherein the calculation is performed on demand.

8. The system of claim 1, wherein the calculation is performed continuously in real time.

9. The system of claim 1, wherein the first electronic position sensor includes a leg length measurement sensor configured to detect a distance between the first electronic position sensor and the second electronic position sensor.

10. The system of claim 1,
wherein the first electronic position sensor includes a laser emitter and the second electronic position sensor includes a receiver; and
wherein the software is configured to determine an offset between the first and second electronic position sensors based on which portion of the receiver detects light from the laser emitter.

11. A method for creating a patient-specific alignment guide, comprising:
receiving data from a scan of a bony anatomic structure of a patient;
creating a 3-dimensional model of the patient's bony anatomic structure from the data;
creating a 3-dimensional model of a patient-specific alignment guide including a surface that is substantially a negative of at least a portion of the patient's bony anatomic structure; and
fabricating the patient-specific alignment guide.

12. The method of claim 11, wherein the bony anatomic structure is a pelvis.

13. The method of claim 12, further comprising determining at least one of an angle of inclination and an angle of forward flexion of the patient-specific alignment guide relative to the patient's pelvis.

14. The method of claim 11, wherein fabricating the patient-specific alignment guide utilizes additive manufacturing.

15. The method of claim 11, wherein the data received is a series of 2-dimensional computed tomography (CT) images.

16. A method for positioning a prosthesis, comprising:
coupling a first electronic position sensor to a bony anatomic structure of a patient;
positioning a patient-specific alignment guide against the bony anatomic structure of the patient, wherein the patient-specific alignment guide includes a surface created from a scan of the patient's bony anatomic structure that is substantially a negative of at least a portion of the patient's bony anatomic structure such that the alignment guide fits against the bony anatomic structure of the patient in only one orientation;
coupling a second electronic position sensor to the patient-specific alignment guide;
communicating position and orientation information from each of the first and second electronic position sensors to a digital data processor;
removing the patient-specific alignment guide;
coupling the second electronic position sensor to an instrument that is coupled to a prosthesis;
displaying one or more calculated angular relationships of the prosthesis relative to the patient's bony anatomic structure based on position and orientation information communicated from each of the first and second electronic position sensors; and
positioning the prosthesis relative to the bony anatomic structure of the patient such that the one or more displayed angular relationships are at desired levels.

17. The method of claim 16, wherein coupling the first electronic position sensor to the patient's pelvis includes driving two pins into the bony anatomic structure in a parallel orientation and sliding two through-holes formed in the first electronic position sensor over the two pins.

18. The method of claim 16, wherein coupling the first electronic position sensor to the patient's pelvis includes driving one pin into the bony anatomic structure and sliding one through-hole formed in the first electronic position sensor over the pin.

19. The method of claim 16, further comprising saving position and orientation information communicated from each of the first and second electronic position sensors when the patient-specific alignment guide is positioned against the bony anatomic structure; and
providing guidance to a user to direct the second electronic position sensor to the saved position relative to the first electronic position sensor when the second electronic position sensor is coupled to the instrument and after the patient-specific alignment guide has been removed.

20. The method of claim 16, further comprising removably securing the patient-specific alignment guide against the bony anatomic structure using at least one surgical pin.

21. The method of claim 16, further comprising coupling a sensor mount to the instrument, wherein the sensor mount is configured to orient the second electronic position sensor at an angle of about 45° relative to a longitudinal axis of the instrument.

22. The method of claim 16, wherein the bony anatomic structure is a pelvis.

23. The method of claim 22, wherein the prosthesis is configured to be inserted into an acetabulum of the patient's pelvis.

24. A method for positioning a prosthesis, comprising:
coupling a first electronic position sensor to a bony anatomic structure of a patient;
coupling a second electronic position sensor to an instrument that is coupled to a prosthesis;
positioning the prosthesis relative to the bony anatomic structure of the patient;
communicating position and orientation information from each of the first and second electronic position sensors to a digital data processor;
displaying one or more calculated angular relationships of the prosthesis relative to the patient's bony anatomic structure based on position and orientation information communicated from each of the first and second electronic position sensors;
receiving by the digital data processor patient-specific information including one or more desired angular relationships; and
adjusting the position of the prosthesis relative to the bony anatomic structure of the patient such that the one or more displayed angular relationships match the desired angular relationships.

25. The method of claim 24, wherein the patient-specific information received by the digital data processor is derived from one or more x-ray images taken intra-operatively after positioning the prosthesis relative to the bony anatomic structure of the patient.
